(19) United States
(12) Patent Application Publication
(10) Pub. No.: US 2007/0135382 A1
(43) Pub. Date: Jun. 14, 2007

(54) Use of peroxisome proliferator-activated receptor gamma (ppary) and/or retinoic acid receptor (rxr) agonists to inhibit platelet functions

(76) Inventors: Richard P. Phipps, Pittsfortd, NY (US); Patricia J. Sime, Pittsford, NY (US); Neil Blumberg, Pittsford, NY (US)

Correspondence Address: NIXON PEABODY LLP - PATENT GROUP, CLINTON SQUARE, P.O. BOX 31051, ROCHESTER, NY, 14603-1051, US

(21) Appl. No.: 10/576,824

(22) PCT Filed: Oct. 22, 2004
(86) PCT No.: PCT/US04/35065

§ 371 Date: Aug. 31, 2006

Related U.S. Application Data
(60) Provisional application No. 60/513,372, filed on Oct. 22, 2003.
(60) Provisional application No. 60/553,657, filed on Mar. 16, 2004.
(60) Provisional application No. 60/567,397, filed on Apr. 30, 2004.

(51) Int. Cl. A61K 31/685; A61K 31/557; A61K 31/426; A61K 31/198; A61K 31/17

(52) U.S. Cl. 514/78; 514/573; 514/369; 514/419; 514/559; 514/567; 514/594

(57) ABSTRACT

Methods of inhibiting mammalian platelet release of CD40 ligand, thromboxanes, or prostaglandin E2, or surface expression of CD40 ligand that involve contacting mammalian platelets with an effective amount of a PPARγ agonist, an RXR agonist, or a combination thereof. As a consequence of inhibiting CD40 ligand and thromboxane release, the present invention allows for inhibition of thrombus fon-nation by (or clotting activities of) activated platelets, as well as treating or preventing CD40 ligand-mediated conditions and/or thromboxane-mediated conditions. Use of PPARγ agonist, RXR agonist, and/or inducers of PPARγ agonist in preparing a stored blood product, and for diagnostic testing of patient samples is also disclosed.

This application claims the priority benefit of U.S. Provisional Patent Applications Ser. Nos. 60/513,372, filed Oct. 22, 2003; 60/553,657, filed Mar. 16, 2004; and 60/567,397, filed Apr. 30, 2004, each of which is hereby incorporated by reference in its entirety.

The present invention was made, at least in part, with funding received from the National Institutes of Health under grant number RO1 DE11390. The U.S. government may retain certain rights in this invention.

### FIELD OF THE INVENTION

The present invention relates generally to the use of PPARγ, PPARγ agonists and/or RXR agonists to inhibit platelet functions, including platelet aggregation, release of CD40 ligand, release of thromboxanes, release of prostaglandins, and surface expression of CD40 ligand. Consequently, the present invention further relates to uses of PPARγ agonists and/or RXR agonists to treat patients for CD40 ligand- or thromboxane-mediated conditions.

### BACKGROUND OF THE INVENTION

Platelet activation is central to the formation of thrombus, which precipitates most unstable coronary syndromes. The angiographic severity of coronary stenoses may not predict the occurrence of acute cardiac events, since rupture of atheromatous plaque and subsequent thrombosis in slightly stenosed vessels may underlie many myocardial infarctions. Normally, the intact endothelium prevents platelet activation, but intimal injury associated with endothelial denudation and plaque rupture exposes subendothelial collagen and von Willebrand factor, supporting prompt platelet adhesion and activation. Local platelet activation then promotes the recruitment of platelets and the formation of thrombus.

The importance of platelet-dependent thrombosis has made activated platelets a common therapeutic target in acute coronary syndromes. Platelet inhibitors have included aspirin, thienopyridines, and glycoprotein IIb/IIIa inhibitors. Although these agents have distinct mechanisms of action, all of them inhibit fibrinogen-dependent platelet-platelet associations.

Acute myocardial infarction is commonly diagnosed by measuring markers of cardiac necrosis. These markers reflect the extent of cardiac damage but fail to provide direct information about plaque disruption or platelet activation. Because the outcome of acute coronary syndromes is highly dependent on inflammation and thrombosis, it is possible that measurement of these two processes will allow better assessment of plaque instability. An established link between inflammation and thrombosis in acute coronary syndromes is the formation of platelet-monocyte (heterotypic) aggregates when platelets bind by way of surface-expressed P-selectin (CD62P) to P-selectin glycoprotein ligand 1, a leukocyte receptor (Rinder et al., “Dynamics of Leukocyte-Platelet Adhesion in Whole Blood,” Blood 78:1730-1737 (1991); Freedman and Loscalzo, “Platelet-Monocyte Aggregates: Bridging Thrombosis and Inflammation,” Circulation 105:2130-2132 (2002)). Circulating platelet-monocyte aggregates have been shown to be an early marker of acute myocardial infarction (Furman et al., “Circulating Monocyte-Platelet Aggregates are an Early Marker of Acute Myocardial Infarction,” J. Am. Coll. Cardiol. 38:1002-1006 (2001)) and to contribute to the formation of atherosclerotic lesions (Huo et al., “Circulating Activated Platelets Exacerbate Atherosclerosis in Mice Deficient in Apolipoprotein E,” Nat. Med. 9:61-67 (2003)).

Although platelet-monocyte aggregates can provide useful information about the thrombotic or inflammatory state and can identify patients at high risk for cardiac events, their measurement can be cumbersome. As compared with platelet-monocyte aggregates, measurement of soluble CD40 ligand (also called CD154), an immunomodulator, does not require flow cytometry and can be accomplished with stored samples. CD40 ligand is a trimeric, transmembrane protein of the tumor necrosis factor family and, together with its receptor CD40, is an important contributor to the inflammatory processes that lead to atherosclerosis and thrombosis (Henn et al., “CD40 Ligand on Activated Platelets Triggers an Inflammatory Reaction of Endothelial Cells,” Nature 391:591-594 (1998)). A large variety of immunologic and vascular cells have been found to express CD40, CD40 ligand, or both. Both CD40 and CD40 ligand have been shown to be present in human atheroma (Schonbeck and Libby P, “CD40 Signaling and Plaque Instability,” Circ. Res. 89:1092-1103 (2001)).

In platelets, CD40 ligand is rapidly translocated to the platelet surface after stimulation and is up-regulated in fresh thrombus (Henn et al., “CD40 Ligand on Activated Platelets Triggers an Inflammatory Reaction of Endothelial Cells,” Nature 391:591-594 (1998)). The surface-expressed CD40 ligand is then cleaved from the platelets over a period of minutes to hours, subsequently generating a soluble fragment (soluble CD40 ligand) (Andre et al., “Platelet-Derived CD40L: The Switch-Hitting Player of Cardiovascular Disease,” Circulation 106:896-899 (2002)). Although it may also be shed from stimulated lymphocytes, it is estimated that more than 95 percent of circulating CD40 ligand is derived from platelets (Andre et al., “Platelet-Derived CD40L: The Switch-Hitting Player of Cardiovascular Disease,” Circulation 106:896-899 (2002)). Soluble CD40 ligand has been shown to be associated with an increased risk of cardiovascular events in apparently healthy women (Schonbeck et al., “Soluble CD40L and Cardiovascular Risk in Women,” Circulation 104:2266-2268 (2001)).

Heeschen et al. provide important information about the clinical relevance of levels of soluble CD40 ligand in patients presenting with chest pain (Heeschen et al., “Soluble CD40 Ligand in Acute Coronary Syndromes,” N. Engl. J. Med. 348:1104-1111 (2003)). In their study, soluble CD40 ligand identified patients at high risk for acute coronary syndromes. In the original CAPTURE report, elevated levels of troponin T identified a subgroup of patients who significantly benefited from treatment with abciximab (“Randomised Placebo-Controlled Trial of Abciximab Before and During Coronary Intervention in Refractory Unstable Angina: The CAPTURE Study,” Lancet 349:1429-1435 (1997); Erratum, Lancet 350:744 (1997)). The current study demonstrates that, in contrast to troponins, the predictive value of the level of soluble CD40 ligand with respect to the effects of abciximab is independent of the presence or absence of recent myocardial infarction. In patients who received placebo, elevated levels of soluble CD40 ligand were associated with a significantly increased risk of death or myocardial infarction. The increased risk associated with elevated levels of soluble CD40 ligand was reduced with abciximab treatment. Taken together, these observations suggest that elevation of soluble CD40 ligand identifies patients with an increased risk of thrombosis. In addition, among patients who were negative for troponin T, soluble CD40 ligand identified those at increased risk for cardiac events, suggesting that measurement of these diagnostic markers of coronary ischemia has additive benefits.

Peroxisome proliferator-activated receptors (PPARs) are members of a nuclear hormone receptor superfamily of ligand-activated transcription factors. There are three PPAR subtypes PPARα, PPARβ/δ and PPARγ. The genes encoding the PPAR subtypes each reside on different chromosomes and have distinct tissue expression patterns (Daynes and Jones, “Emerging Roles of PPARs in Inflammation and Immunity,” Nature Rev. Immunol. 2:748-759 (2002)). While many reports focus on PPAR expression in the nucleus, PPARγ, in particular, is also found in the cytoplasm (Padilla et al., “Human B Lymphocytes and B Lymphomas Express PPAR-γ and Are Killed by PPAR-γ Agonists,” Clinical Immunology 103:22-33 (2002); Kelly et al., “Commensal Anaerobic Gut Bacteria Attenuate Inflammation by Regulating Nuclear-Cytoplasmic Shuttling of PPAR-γ and Rel A,” Nat. Immunol. 5:104-112 (2004)).

PPARγ is highly expressed in white adipose tissue and was initially described as being important for regulating gene expression in metabolism, insulin responsiveness, and adipocyte differentiation (Spiegelman et al., “PPAR gamma and the Control of Adipogenesis,” Biochemie. 79:111-112 (1997); Fajas et al., “The Organization, Promoter Analysis and Expression of the Human PPARγ Gene,” J. Biol. Chem. 272:18779-18789 (1997)). While PPARγ was originally thought to be found mainly in fat tissue, it is in fact widely expressed by many types of cells including macrophages, B and T lymphocytes, epithelial, endothelial, smooth muscle, and fibroblastic cells (Padilla et al., “Human B Lymphocytes and B Lymphomas Express PPAR-γ and Are Killed by PPAR-γ Agonists,” Clinical Immunology 103:22-33 (2002); Ricote et al., “The Peroxisome Proliferator-Activated Receptor-Gamma is a Negative Regulator of Macrophage Activation,” Nature 391:79-82 (1998); Harris and Phipps, “Prostaglandin D₂, its Metabolite 15-d-PGJ₂, and Peroxisome Proliferator Activated Receptor-Gamma Agonists Induce Apoptosis in Transformed, but not Normal, Human T Lineage Cells,” Immunology 105:23-34 (2002); Su et al., “A Novel Therapy for Colitis Utilizing PPAR-Gamma Ligands to Inhibit the Epithelial Inflammatory Response,” J. Clin. Invest. 104:383-389 (1999); Marx et al., “PPAR Gamma Activation in Human Endothelial Cells Increases Plasminogen Activator Inhibitor Type-1 Expression: PPAR Gamma as a Potential Mediator in Vascular Disease,” Arteriosclerosis, Thrombosis & Vascular Biology 19:546-551 (1999); Iijima et al., “Expression of Peroxisome Proliferator-Activated Receptor Gamma (PPAR Gamma) in Rat Aortic Smooth Muscle Cells,” Bioch. Biophys. Res. Comm. 247:353-356 (1998); Lee et al., “Peroxisome Proliferation, Adipocyte Determination and Differentiation of C3H10T1/2 Fibroblast Cells Induced by Humic Acid: Induction of PPAR in Diverse Cells,” J. Cell. Physiol. 179:218-25 (1999)). PPARγ has also come to prominence as PPARγ agonists play an important role in immune function by dampening inflammation, by attenuating macrophage/monocyte synthesis of proinflammatory cytokines, and by inducing apoptosis in B lymphocytes (Jiang et al., “PPAR-Gamma Agonists Inhibit Production of Monocyte Inflammatory Cytokines,” Nature 391:82-86 (1998); Ricote et al., “The Peroxisome Proliferator-activated Receptor-gamma is a Negative Regulator of Macrophage Activation,” Nature 391:79-82 (1998); Padilla et al., “Human B Lymphocytes and B Lymphomas Express PPAR-γ and Are Killed by PPAR-γ Agonists,” Clinical Immunology 103:22-33 (2002); Padilla et al., “Peroxisome Proliferator Activator Receptor-γ Agonists and 15-Deoxy-Δ^{12,14}-PGJ₂ Induce Apoptosis in Normal and Malignant B-Lineage Cells,” J. Immunol. 165:6941-6948 (2000)). PPARγ has also emerged as a key target for malignant cells, as PPARγ agonists have shown therapeutic potential for B lymphoma and various epithelial-derived cancers (Padilla et al., “Human B Lymphocytes and B Lymphomas Express PPAR-γ and Are Killed by PPAR-γ Agonists,” Clinical Immunology 103:22-33 (2002); Jackson et al., “Potential Role for Peroxisome Proliferator Activated Receptor (PPAR) in Preventing Colon Cancer,” Gut 52:1317-1322 (2003); Mueller et al., “Terminal Differentiation of Human Breast Cancer Through PPAR Gamma,” Molecular Cell. 1:465-470 (1998)).

Megakaryocytes are the biggest cell of the bone marrow and the parent cell of platelets. Platelets are derived from the cytoplasm of megakaryocytes and are released to the bloodstream under the effects of cytokines such as IL-6 and IL-11 (Teramura et al., “Interleukin-11 Enhances Human Megakaryocytopoiesis in vitro,” Blood 79:327-331 (1992); Burstein et al., “Thrombocytopoiesis in Normal and Sublethally Irradiated Dogs: Response to Human Interleukin-6,” Blood 80:420-428 (1992)). Platelets are nuclear cells that have a plasma membrane, surface-connected canalicular and tubular system, mitochondria, granules, lysosomes, and peroxisomes (Bentfeld-Barker and Bainton, “Identification of Primary Lysosomes in Human Megakaryocytes and Platelets,” Blood 59:472-481 (1982)). Recent studies demonstrate that platelets and many of their products are not only important in hemostasis, but have now emerged as important in immunoregulation and inflammation. For example, platelets produce key inflammatory mediators such as transforming growth factor-β (TGF-β), thromboxane A₂, and PGE₂ (Scheuerer et al., “The CXC-chemokine Platelet Factor 4 Promotes Monocyte Survival and Induces Monocyte Differentiation into Macrophages,” Blood 95:1158-1166 (2000); Gear et al., “Platelet Chemokines and Chemokine Receptors: Linking Hemostasis, Inflammation, and Host Defense,” Microcirculation 10:335-350 (2003); Vezza et al., “Prostaglandin E2 Potentiates Platelet Aggregation by Priming Protein Kinase C,” Blood 82:2704-2713 (1993)). The recent key demonstration that activated human platelets express and expel CD40 ligand (CD40L, formally known as CD154) provides a mechanism of interaction with CD40 expressing cells that include macrophages and vascular structural cells (Phipps, “Atherosclerosis: The Emerging Role of Inflammation and the CD40-CD40Ligand System,” Proc. Natl. Acad. Sci. USA 97:6930-6932 (2000); Phipps et al., “Platelet Derived CD154 (CD40 Ligand) and Febrile Responses to Transfusion,” Lancet 357:2023-2024 (2001); Danese et al., “Platelets Trigger a CD40-Dependent Inflammatory Response in the Microvasculature of Inflammatory Bowel Disease Patients,” Gastroenterolog 124:1249-1264 (2003); Henn et al., “CD40 Ligand on Activated Platelets Triggers an Inflammatory Reaction of Endothelial Cells,” Nature 391:591-594 (1998)). These cells when activated through CD40 express Cox-2 and prostaglandins, adhesion molecules, and cytokines such as IL-6 and tissue factor (Mach et al., “CD40 Signaling in Vascular Cells: A Key Role in Atherosclerosis?” Atherosclerosis 137:S89-95 (1998); Linton and Fazio, “Cyclooxygenase-2 and Atherosclerosis,” Curr. Opin. Lipidology 13:497-504 (2002)). Many new studies now demonstrate that elevated CD40L levels in blood are associated with acute coronary syndromes and stroke (Heeschen et al., “Soluble CD40L in Acute Coronary Syndromes,” New Engl. J. Medicine 348:1104-1111 (2003)). Interestingly, elevated serum levels of CD40L predict an increased cardiovascular risk in a healthy population (Schonbeck et al., “Soluble CD40L and Cardiovascular Risk in Women,” Circulation. 104:2266-2268 (2001)).

The present invention relates to the surprising findings that human megakaryocytes and platelets express PPARγ, and are susceptible to PPARγ agonists and RXR agonists that dampen proinflammatory and proatherogenic platelet functions, including platelet aggregation, platelet release of CD40 ligand, release of thromboxanes, release of prostaglandins, and surface expression of CD40 ligand.

### SUMMARY OF THE INVENTION

A first aspect of the present invention relates to a method of inhibiting release of CD40 ligand, thromboxanes, or prostaglandin E2, or inhibiting CD40 ligand surface expression, by mammalian platelets. This aspect of the present invention includes the step of contacting mammalian platelets with an effective amount of PPARγ, a PPARγ agonist, an RXR agonist, or a combination thereof, whereby said contacting inhibits release of CD40 ligand, thromboxanes, prostaglandin E2, or a combination thereof, and/or inhibits CD40 ligand surface expression by the mammalian platelets.

A second aspect of the present invention relates to a method of inhibiting thrombosis. This aspect of the present invention includes the step of contacting mammalian platelets with an effective amount of PPARγ, a PPARγ agonist, an RXR agonist, or a combination thereof, whereby said contacting inhibits formation of a thrombosis by the mammalian platelets.

A third aspect of the present invention relates to a method of treating or preventing a thrombotic condition or disorder. This aspect of the invention includes the step of contacting mammalian platelets, in an individual exhibiting symptoms of or predisposed to a thrombotic condition or disorder, with an effective amount of PPARγ, a PPARγ agonist, an RXR agonist, or a combination thereof, whereby said administering inhibits platelet activation to treat or prevent the thrombotic condition or disorder.

A fourth aspect of the present invention relates to a method of improving the quality of a blood product. This aspect of the invention includes the steps of providing PPARγ, a PPARγ agonist, an RXR agonist, an inducer of a PPARγ agonist, or a combination thereof; and introducing PPARγ, the PPARγ agonist, the RXR agonist, the inducer of a PPARγ agonist, or the combination thereof, to a blood product, wherein the PPARγ agonist, the RXR agonist, the inducer of a PPARγ agonist, or the combination thereof inhibits clotting or activation of platelets in the blood product and thereby improves the quality thereof.

A fifth aspect of the present invention relates to a stored blood product that includes: a blood product that contains platelets and an amount of PPARγ, a PPARγ agonist, an RXR agonist, an inducer of a PPARγ agonist, or a combination thereof that is effective to inhibit platelet activation.

A sixth aspect of the present invention relates to a method of inhibiting platelet aggregation. This aspect of the present invention includes the step of contacting mammalian platelets with an effective amount of PPARγ, a PPARγ agonist, an RXR agonist, or a combination thereof, whereby said contacting inhibits aggregation of the mammalian platelets.

A seventh aspect of the present invention relates to a method of treating or preventing a CD40 ligand-mediated or thromboxane-mediated condition. This aspect of the invention includes the step of contacting platelets, in an individual exhibiting or predisposed to a CD40 ligand-mediated or thromboxane-mediated condition, with PPARγ, a PPARγ agonist, an RXR agonist, an inducer of a PPARγ agonist, or a combination thereof, whereby said contacting inhibits the release of CD40 ligand and/or thromboxane by platelets, thereby treating or preventing the CD40 ligand-mediated or thromboxane-mediated condition.

An eighth aspect of the present invention relates to a method of assessing the activity of a compound as a PPARγ agonist. This aspect of the invention includes the steps of: combining a compound with both platelets and a platelet activator; determining the level of CD40 ligand or thromboxane released from the platelets; and comparing the level of CD40 ligand or thromboxane released from the platelets to the level of CD40 ligand or thromboxane released from a standard, wherein deviation from the standard, or absence thereof, indicates activity of the compound as a PPARγ agonist.

A ninth aspect of the present invention relates to a method of diagnosing a CD40 ligand-mediated condition. This aspect of the invention includes the steps of: obtaining a patient sample; and determining the level of PPARγ in the patient sample, wherein a reduced (or lower than normal) PPARγ level indicates the presence of a CD40 ligand-mediated condition.

A tenth aspect of the present invention relates to a method of assessing the efficacy of a PPARγ agonist therapy. This aspect of the invention includes the steps of: obtaining a patient sample, the patient having been previously administered a PPARγ agonist or an inducer of a PPARγ agonist for treating a medical condition or disorder; and determining the level of PPARγ in the patient sample, wherein an elevated PPARγ level, relative to a baseline PPARγ level for the patient prior to said administration, indicates the efficacy of the PPARγ agonist therapy.

An eleventh aspect of the present invention relates to a method of treating or preventing a CD40 ligand-mediated condition. This aspect of the invention includes the step of: treating a patient exhibiting or predisposed to a CD40 ligand-mediated condition with recombinant PPARγ, whereby said treating inhibits the release of CD40 ligand by platelets, thereby treating or preventing the CD40 ligand-mediated condition.

A twelfth aspect of the present invention relates to a method of modifying megakaryocytes. This aspect of the present invention includes the step of exposing a megakaryocyte to PPARγ, a PPARγ agonist, an RXR agonist, an inducer of a PPARγ agonist, or a combination thereof, whereby said exposing phenotypically modifies the megakaryocyte to produce daughter platelets that minimize pro-inflammatory and/or prothrombotic responses by the platelets.

A thirteenth aspect of the present invention relates to a method of treating or preventing a CD40 ligand-mediated or thromboxane-mediated condition. This aspect of the present invention includes the step of treating a patient exhibiting or predisposed to a CD40 ligand-mediated condition with recombinant PPARγ, whereby said treating inhibits the release of CD40 ligand and/or thromboxane by platelets, thereby treating or preventing the CD40 ligand-mediated or thromboxane-mediated condition.

The applicants have identified the presence of PPARγ in platelets and demonstrated its role in inhibiting platelet aggregation, inhibiting release of CD40 ligand, thromboxanes, and prostaglandins, as well as inhibiting expression of CD40 ligand on the platelet surface, all of which are known factors implicated in the development of pro-inflammatory and thrombotic conditions or disorders. Approximately ninety-five percent of circulating CD40 ligand exists in platelets (see André et al., “Platelet-Derived CD40L: The Switch-Hitting Player of Cardiovascular Disease,” Circulation 106:896-899 (2002), which is hereby incorporated by reference in its entirety). Thus, the use of PPARγ agonist to blunt the release of CD40 ligand or thromboxanes by platelets can effectively control the level of soluble CD40 ligand or thromboxanes that are present in an individual's circulatory system. In effect, by controlling CD40 ligand, thromboxanes and/or prostaglandins, PPARγ, the PPARγ agonist, or inducers of PPARγ agonist, can be used to treat or prevent development of conditions or disorders mediated by CD40 ligand, thromboxanes, or prostanglandins, including thrombotic conditions or disorders.

### BRIEF DESCRIPTION OF THE DRAWINGS

FIGS. 1A-D demonstrate that PPARγ protein is expressed in the human megakaryoblast cell line, Meg-01, and by human platelets. FIG. 1A is a Western blot of Meg-01 cell line (15 μg) using a polyclonal anti-PPARγ antibody (Calbiochem). PPARγ bands co-migrated with the human adipose tissue protein extract (15 μg) used as positive control. FIGS. 1B-C are Western blots for PPARγ using platelet cell lysates (15 μg) from rigorously purified single donor or pooled platelets. FIG. 1B was prepared using monoclonal anti-PPARγ antibody (Santa Cruz); FIG. 1C was prepared using a polyclonal anti-PPARγ antibody (Calbiochem). Human adipose tissue protein extract (5 μg) was used as positive control (first lanes). The PPARγ protein was shown for three different single donor and pooled platelets samples. Purified human red blood cells (30 μg) are negative for PPARγ (FIG. 1B). Data are representative of more than five experiments. FIG. 1D shows that purified mouse platelets (5 μg) also express PPARγ (lane 1, adipose tissue; lane 2, mouse macrophage cell line; lanes 3 & 4, purified platelets from two different mice).

FIGS. 2A-D demonstrate PPARγ expression in human megakaryoblast cells and platelets. Immunocytochemistry was performed with a rabbit polyclonal anti-PPARγ antibody as described in the Examples. Non-specific staining was assessed using a rabbit IgG isotype control. FIG. 2A shows that nucleated cells and enucleated platelet-like cells of the Meg-01 cell line were stained for PPARγ. Meg-01 cells stain in the nucleus and the cytoplasm. Results were repeated four times with separate preparations of Meg-01 cells. Original magnification is X600. FIG. 2B shows that human platelets express PPARγ. The staining pattern for PPARγ is throughout the platelets. Data are representative of 4 different donor platelet experiments with similar results. Original magnification is X1000. FIG. 2C shows the flow cytometric analysis for intracellular expression of PPARγ in human platelets. Purified platelets were washed and stained with a monoclonal FITC-labeled anti-PPARγ antibody (open histogram) or FITC-labeled IgG1 isotype control (shaded histogram) as described in the Examples. Forward and side scatter gates were set to analyze only platelets. This experiment was repeated three times with similar results. FIG. 2D shows the immunocytochemistry of human bone marrow megakaryocyte for PPARγ. Left panel shows a Diff-Quick staining of a human bone marrow megakaryocyte. Immunohistochemistry was performed with a mouse monoclonal PPARγ antibody as described in the Examples. PPARγ expression is shown in the right panel. Mouse IgG1 isotype control was also used to show non-specific staining (middle panel). In addition to PPARγ immunostaining, light counterstaining was performed with Hematoxylin to visualize the cells. The arrows are pointing at human megakaryocytes. Original magnification is X600. Data are representative of four experiments from four patients with similar results.

FIG. 3 is a gel electrophoresis of reverse transcription polymerase chain reaction products, demonstrating that the human megakaryocyte cell line, Meg-01, but not human platelets express PPARγ mRNA. Total RNA was isolated from Meg-01 cells (lane 6) and human platelets (lanes 3-5) and reverse transcribed into cDNA. The cDNA was amplified with primers specific for β-actin (539 bp product, as a control) or PPARγ (360 bp product). A 100 bp ladder was loaded in lane 1. Human adipose tissue (lane 2) and the human monocyte cell line (THP1) (lane 7) were used as positive controls. Platelet samples were from a single donor (lane 3) or pooled from several donors (lanes 4 and 5). Reverse transcriptase (−) controls were negative in all cases.

FIGS. 4A-C demonstrate that Meg-01 cells and human platelets contain PPARγ that binds the PPARγ DNA consensus sequence. FIG. 4A shows that 15d-PGJ₂ and ciglitazone induce DNA binding of PPARγ protein in Meg-01 cells. After treatment with 15d-PGJ₂ (lane 3) or ciglitazone (lane 4) or DMSO (vehicle control)(lane 2 ), an electrophoretic mobility shift assay (EMSA) was performed. Lane 1 was loaded with free probe (no lysate), and lane 5 was loaded with nuclear extract from 15d-PGJ₂ treated cells incubated with unlabeled probe (cold competitor) as a control for binding specificity. Lane 6 shows the locations of shifted and supershifted PPARγ (supershift with an anti-PPARγ antibody). Shift assays were repeated three times with similar results. FIG. 4B shows via EMSA that platelets have PPARγ DNA binding activity. Platelet extracts were prepared without any treatment from three different pooled platelets as described in the Examples. Lane 1 shows radioactive-labeled probe. Fifty μg of cell extracts were incubated with ³²P-labeled PPARγ oligonucleotides (lane 2, 3, and 4) or cold competitor (unlabeled probe) (lane 5, 6, 7), and then run on a 4% nondenaturing gel. Lanes 8, 9 and 10 indicate the locations of supershifted bands with anti-PPARγ antibody. FIG. 4C shows via transAM® solid phase PPARγ DNA binding activity measurements that platelets have some active DNA binding PPARγ without treatment with PPARγ agonist. However, exposure to PPARγ agonist (20 μM 15d-PGJ₂, ciglitazone, rosiglitazone) significantly enhances binding to the PPARγ DNA response element. Assay background in this experiment was 0.02 OD.

FIG. 5 shows that human platelets express RXR protein. Rigorously purified human platelets were lysed (5 μg protein), and their probed with an anti-RXR antibody by western blot. Platelets do express RXR, as shown in lanes 1-3. Human adipose tissue was used as positive control.

FIGS. 6A-B illustrate the effects of PPARγ agonists on blocking platelet release of CD40L and thromboxane. Purified human platelets were exposed to buffer or with 20 μM 15d-PGJ₂ or rosiglitazone for 15 minutes. The platelets were then activated with 0.8 U/ml thrombin and the supernatants collected at the times shown. Specific ELISA and enzyme immunoassays for CD40L (FIG. 6A) and TXB₂ (FIG. 6B) levels were performed. The increase in supernatant CD40L over time was statistically significant after thrombin activation compared with untreated or PPARγ agonist pretreated samples (p=0.0006 by the log rank test) (FIG. 6A). There were no significant differences in CD40L release when comparing untreated samples to those treated with PPARγ agonist and thrombin. Mean±SD are shown. The increase in supernatant TXB₂ over time was statistically significant after thrombin activation compared with untreated or PPARγ agonist and thrombin treated platelets (p=0.0004 by the log rank test) (FIG. 6B). These data are representative of three separate experiments. Values with an asterisk (*) are significantly different from those treated with 15d-PGJ₂ or rosiglitazone.

FIG. 7 illustrates the ability of PPARγ agonists to block the thrombin-induced increase in platelet surface CD40L expression. Purified human platelets were exposed to 20 μM 15d-PGJ₂ or rosiglitazone for 15 minutes and were then stimulated with 0.8 U/ml thrombin for 60 minutes. The platelets were then stained and prepared for flow cytometry with a monoclonal anti-human CD40L antibody or with control isotype antibody. The graph shows a representative experiment with the results presented as the percent of surface CD40L positive platelets.

FIGS. 8A-B illustrate the effects of PPARγ agonists on platelet function. ATP release from platelets during aggregation was characterized by Lumi-Aggregometry after stimulation with thrombin (1 U/ml) or ADP (5 μM). Both the magnitude and rate of ATP release were reduced (FIG. 8A). The rate of release with normal platelets was defined as 100%. 15d-PGJ₂ (20 μM) resulted in a significant (P=0.002) reduction in the rate of release with thrombin and a similar reduction with ADP (5 μM/L) (P=0.05). The results represent mean±SEM of three experiments. FIG. 8B illustrates the results of a typical experiment showing platelet aggregation with normal control (ADP as platelet agonist) (left panel) compared to 15d-PGJ₂ and rosiglitazone (10 μM) (right panel). Both of these PPARγ agonists decrease the aggregation slope (rate). These findings were reproducible with platelets from several donors.

FIG. 9 is a graph illustrating the effects of rosiglitazone (Avandia), a PPARγ agonist, in attenuating the ability of the platelet activator epinephrine to induce a clot. Human blood was exposed to rosiglitazone or vehicle alone followed by testing in the PFA-100 activated with epinephrine. Blood exposed to rosiglitazone takes longer to form a closure (p=0.019).

FIG. 10 is a graph illustrating the effects of the PPARγ agonist 15d-PGJ₂ in attenuating the ability of the platelet activator ADP to induce a clot. Human blood was exposed to 15d-PGJ₂ or vehicle only followed by testing in the PFA-100 activated with ADP. Blood exposed to rosiglitazone takes longer to form a closure (p=0.012).

FIG. 11 illustrates the effects of PPARγ agonists on blocking platelet release of prostaglandin E₂ (PGE₂). Purified human platelets were exposed to buffer or with 20 μM 15d-PGJ₂ or rosiglitazone for 15 minutes. The platelets were then activated with 0.8 U/ml thrombin and the supernatants collected at the times shown. An enzyme immunoassay for PGE₂ levels was performed. The increase in supernatant PGE₂ over time was statistically significant after thrombin activation compared with PPARγ agonist pretreated samples (p=0.01).

FIGS. 12A-B illustrate the detection of PPARγ in human and mouse tissue and fluid samples as detected by Western blot using monoclonal or polyclonal antibodies that react with PPARγ.

FIG. 13 shows the postulated pathways for natural and synthetic PPARγ small molecule agonists to bind and activate PPARγ and to regulate transcription. The figure also reveals that there are potential PPARγ independent pathways whereby PPARγ agonists can function. PPRE=PPAR response element in DNA that leads to gene transcription.

FIG. 14 schematically illustrates findings linking Diabetes to PPARγ, platelet activation, thrombosis and inflammation. The figure shows that human platelets become activated to express surface CD40L, to aggregate, to release CD40L, as well as other proinflammatory and prothrombotic mediators. Note that exposure of platelets to PPARγ agonists blunts their activation, aggregation and release of proinflammatory and prothrombotic mediators. The end result should be a dampening of thrombosis and inflammation in diabetic and non-diabetic subjects.

### DETAILED DESCRIPTION OF THE INVENTION

The present invention generally involves inhibiting mammalian 5 platelet release of CD40 ligand, thromboxanes, and prostaglandin E2, as well as inhibiting platelet surface expression of CD40 ligand, by contacting the mammalian platelets with PPARγ, a PPARγ agonist (or inducer thereof), an RXR agonist, or a combination thereof. As a consequence of inhibiting CD40 ligand and thromboxane release, the present invention allows for reducing platelet activation and therefore inhibiting platelet aggregation and clot formation; inhibiting thrombus formation by activated platelets, as well as treating or preventing CD40 ligand-mediated conditions and/or thromboxane-mediated conditions. In addition to inhibiting CD40 ligand release by platelets, the present invention also contemplates inhibiting CD40 ligand release and/or synthesis by megakaryocytes, the cells responsible for production of platelets.

The mammalian platelets, whose release of CD40 ligand or thromboxane stores can be inhibited, can be any mammalian platelet that expresses PPARγ. Preferred mammalian platelets are human platelets, although other mammalian platelets, such as those from dogs, cats, horses, cows, pigs, other primates, etc. can also be treated in accordance with the present invention. The mammalian platelets, when contacted in accordance with the present invention, can be located in vitro, ex vivo, or in vivo (i.e., in a patient to be treated in accordance with the present invention). As used herein, the terms “patient” and “individual” refer to any mammal whose platelets contain CD40 ligand or thromboxane stores. In certain embodiments, the patient or individual to be treated can be a diabetic patient susceptible to CD40-ligand mediated conditions or thromboxane-mediated conditions. In other embodiments, the patient or individual to be treated is non-diabetic.

When PPARγ is used in accordance with the present invention, the PPARγ can be either substantially purified PPARγ (i.e., purified from mammalian tissue) or recombinant PPARγ. Recombinant human PPARγ is commercially available from Calbiochem Corp./EMD Biosciences (San Diego, Calif.). Alternatively, fragments thereof that are capable of blunting platelet release of CD40 ligand, thromboxanes, or PGE₂ can also be used. Fragments possessing one or more domains can be used, such as those possessing one or more of the ligand binding domain, DNA binding domain, RXR dimerization domain, or co-activator interacting domain(s).

PPARγ agonists are agents that bind to PPARγ and activate receptor-activated pathways. The PPARγ agonists can optionally have dual activity on other PPAR receptors (PPARα and PPARδ). Exemplary PPARγ agonists include, without limitation, cyclopentenone class prostaglandins, thiazolidinediones, glitazones, lysophosphatidic acid (“LPA”) or LPA derivatives (McIntyre et al., “Identification of an intracellular receptor for lysophosphatidic acid (LPA): LPA is a transcellular PPAR gamma agonist,” Proc. Natl. Acad. Sci. USA 100:131-136 ; (2003), which is hereby incorporated by reference in its entirety), tyrosine-based agonists, indole-derived agonists, and combinations thereof. A preferred member of the cyclopentenone class of prostaglandins is 15D-prostaglandin J₂. Preferred thiazolidinediones and/or glitazones include, without limitation, ciglitazone, troglitazone, pioglitazone, rosiglitazone, SB213068 (Smith Kline Beecham), GW1929, GW7845 (Glaxo-Wellcome), and L-796449 (Merck). Suitable tyrosine-based agonists include N-(2-benzylphenyl)-L-tyrosine compounds (Henke et al., N-(2-benzylphenyl)-L-tyrosine PPARgamma Agonists: Discovery of a Novel Series of Patent Antihyperglycemic and Antihyperlipidemic Agents,” J. Med. Chem. 41:5020-5036 (1998), which is hereby incorporated by reference in its entirety. Suitable indole-derived agonists include those disclosed, e.g., in Hanks, et al., “Synthesis and Biological Activity of a Novel Series of Indole-derived PPARgamma Agonists,” Biorg. Med. Chem LLH. 9(23):3329-3334 (1999), which is hereby incorporated by reference in its entirety. Any other PPARγ agonists, whether now known or hereafter developed, can also be utilized in accordance with the present invention.

In addition to the use of PPARγ agonists per se, inducers of PPARγ agonists can also be utilized in accordance with the present invention. Inducers of PPARγ agonists are agents that induce an increase in the expression or production of a native PPARγ agonist. Exemplary inducers of PPARγ agonists include, without limitation, decorin or fragments thereof, enzymes that catalyze formation of prostaglandin D₂ precursor, and combinations thereof. Decorin is a small chondroitin/dermatan sulphate proteoglycan that binds the cytoline transforming growth factor beta (TGF-β) through its core protein. Preferred enzymes that catalyze formation of prostaglandin D₂ precursor are hematopoiefic prostaglandin-D synthase and a lipocalin-form prostaglandin-D synthase. Any other inducers of PPARγ agonists, whether now known or hereafter developed, can also be utilized in accordance with the present invention.

RXR agonists are agents that bind to the retinoic acid receptor and activate receptor-activated pathways. Exemplary RXR agonists include, without limitation, 9-cis-retinoic acid, trans-retinoic acid, any synthetic RXR agonists, e.g., those available from Ligand Pharmaceuticals (San Diego, Calif.), and combinations thereof. Any other RXR agonists, whether now known or hereafter developed, can also be utilized in accordance with the present invention.

In addition to the above agents, PPARα or PPARδ agonists can also be used in combination with the other agents described above. PPARα agonists are agents that bind to PPARα and activate receptor-activated pathways, and PPARδ agonists are agents that bind to PPARδ and activate receptor-activated pathways. A number of known agonists have dual receptor activity. Any PPARα agonists or PPARδ agonists, whether now known or hereafter developed, can also be utilized in accordance with the present invention.

A number of in vitro and ex vivo uses are expected with the present invention, including stored blood products and treatment of blood outside of the body for its immediate return, such as in the case of a dialysis machine or a heart-lung machine. Basically, any in vitro or ex vivo activity involving storage or handling of blood products can be enhanced in accordance with the present invention.

The stored blood products of the present invention include (i) an amount of PPARγ, a PPARγ agonist, an RXR agonist, an inducer of a PPARγ agonist, or a combination thereof that is effective to inhibit platelet activation; and (ii) any blood product that contains platelets. The PPARγ, a PPARγ agonist, an RXR agonist, an inducer of a PPARγ agonist, or a combination thereof is intended to be supplied exogenously. That is, with respect to PPARγ or naturally occurring inducers of PPARγ, they are added to the stored blood product in addition to any quantities that may naturally be present therein. The stored blood product can also contain an anticoagulant or other agents, e.g., PPARα agonists. Exemplary stored blood products include, without limitation, whole blood, plasma, white blood cell products, and concentrated platelets.

It is expected that storage of blood products with PPARγ, a PPARγ agonist, an RXR agonist, or a combination thereof will improve the quality of stored blood products (as compared to similarly stored blood products lacking such an additive) and, as a result, may enhance the shelf-life of the stored blood product. This aspect of the present invention generally involves introducing to a blood product either PPARγ, a PPARγ agonist, an RXR agonist, or a combination thereof, wherein the introduced agents inhibit clotting or activation of platelets in the blood product and thereby improve the quality thereof.

The introducing of the above-identified agents to the blood product is preferably carried out prior to any storage of the blood product. For whole blood, it is therefore desirable to collect whole blood from a patient or donor into a receptacle that already contains one or more of the above-identified agents. For blood fractions (such as plasma or concentrated platelets), it is possible to introduce those agents to either the whole blood prior to separation of blood fractions therefrom or to the blood fraction after its separation.

With respect to treatment of whole blood outside of the body (and prior to its return), it is possible to introduce one or more of the above-identified agents to the whole blood for purposes of inhibiting platelet activation and aggregation, which normally occurs during and following the ex vivo blood treatment procedure.

Suitable dosages for in vitro and ex vivo uses include doses between about 1 μM and about 100 μM, preferably between about 1 μM to about 50 μM, more preferably between about 1 μM to about 10 μM.

For in vivo applications of the present invention, administration of the PPARγ agonist or the inducer of a PPARγ agonist to a mammal can be achieved in a manner that achieves a desired reduction in the release of pro-inflammatory and pro-thrombotic modulators, such as CD40 ligand, thromboxanes, and PGE2. In particular, it is desirable to provide for at least a 10 percent reduction in soluble CD40 ligand or thromboxanes (inactivie form A₂ or active form B₂) present in patient samples, preferably at least a 25 percent reduction, more preferably at least a 50 percent reduction in soluble CD40 ligand or thromboxanes. In alternative embodiments, higher reductions in CD40 ligand and/or thromboxane levels are contemplated. Although any one or more routes of administration can be utilized, preferred modes of administration include, without limitation, topical application, intranasal instillation, inhalation, intravenous injection, intra-arterial injection, intramuscular injection, application to a wound site, application to a surgical site, intracavitary injection, by suppository, subcutaneously, intradermally, transcutaneously, by nebulization, intraplurally, intraperitoneally, intraventricularly, intra-articularly, intra-aurally, intraocularly, or intraspinally.

As a result of the in vivo treatment to inhibit release of the pro-inflammatory modulators by platelets, the present invention also affords a method of inhibiting the activation of platelets, inhibiting the aggregation of activated platelets, and inhibiting the formation of clots that contain activated platelets. Consequently, the present invention likewise affords a method of treating or preventing thrombotic conditions or disorders.

Thrombotic conditions or disorders to be treated or prevented can include one or more of stroke, venous or arterial thrombosis, disseminated intravascular coagulation, myocardial infarction, pulmonary thrombo-embolism, and pulmonary hypertension (primary or secondary). Any of the above listed agents, including PPARγ, PPARγ agonists, RXR agonists, inducers of a PPARγ agonist, or combinations thereof, can be administered (as noted above) to treat or prevent the thrombotic condition or disorder. Patients to be treated in accordance with this aspect of the present invention can be those exhibiting symptoms of or predisposed to a thrombotic condition or disorder. Symptoms of thrombotic conditions or disorders can include, without limitation, pain, numbness, loss of function, swelling, bleeding, weakness, arrhythmia, pallor, shortness of breath, dysphasia, aphasia, dysarithyia, visual loss, paresis, hearing loss, bruising, and syncope. Persons predisposed to thrombotic conditions or disorders are those patients currently asymptomatic, and can include those having a family history of such conditions or disorders or having had prior treatment for such conditions or disorders.

As a result of the in vivo treatment to inhibit CD40 ligand and thromboxane release by platelets, and CD40 ligand expression by platelets, the present invention also affords the treatment or prevention of a CD40 ligand- or thromboxane-mediated conditions.

CD40 ligand has been implicated in a number of diseases or disorders including, without limitation, diabetes, atherosclerosis, induced multiple sclerosis, venous or arterial thrombosis, pulmonary fibrosis, systemic lupus erythematous, renal fibrosis, hepatic cirrhosis, cerebral gliosis, disseminated intravascular coagulation, myocardial infarction, pulmonary thrombo-embolism, and pulmonary hypertension. Any PPARγ agonist, RXR agonist, inducer of a PPARγ agonist, or combinations thereof can be administered (as noted above) to treat or prevent the CD40 ligand-mediated condition. Patients to be treated in accordance with this aspect of the present invention can be those exhibiting symptoms of or predisposed to CD40 ligand-mediated condition. Symptoms of CD40 ligand-mediated conditions can include, without limitation, shortness of breath, cough, edema or swelling (both generally and particularly in the legs), chest pain, limb weakness, claudication, polyurea, polydipsia, bruising, bleeding, limb pain, and abdominal pain and swelling. Persons predisposed to CD40 ligand-mediated conditions are those patients currently asymptomatic, and can include those having a family history of such conditions or disorders or having had prior treatment for such conditions or disorders.

Suitable dosage levels include those capable of achieving blood levels of about 1 μM up to about 1 mM, preferably about 1 μM up to about 500 μM, more preferably about 1 μM up to about 100 μM.

Thromboxanes A₂ and B₂ have been implicated in a number of diseases or disorders including, without limitation, diseases of coagulation, asthma, anti-phospholipid syndrome, and those involving chronic inflammation (e.g., Rheumatid arthritis). Any PPARγ agonist, RXR agonist, inducer of a PPARγ agonist, or combinations thereof can be administered (as noted above) to treat or prevent the thromboxane-mediated condition. Patients to be treated in accordance with this aspect of the present invention can be those exhibiting symptoms of or predisposed to thromboxane-mediated conditions. Symptoms of thromboxane-mediated conditions can include, without limitation, thrombosis, asthma, and those associated with anti-phospholipid syndrome. Persons predisposed to thromboxane-mediated conditions are those patients currently asymptomatic, and can include those having a family history of such conditions or disorders or having had prior treatment for such conditions or disorders.

For each of the above-identified in vivo uses, the treatment of pre-existing conditions relates to controlling the severity of symptoms associated with the condition. That is, symptoms can be maintained (i.e., no worsening) or improved, either substantially or wholly, with continued administration. By preventing a condition, it is intended that development of the condition or onset of the associated symptoms can be delayed or avoided, either substantially or wholly, with continued administration.

As an alternative to administering PPARγ, a PPARγ agonist, an RXR agonist, or an inducer of a PPARγ agonist, recombinant DNA techniques can be utilized in a gene therapy approach, particularly for treating chronic conditions that are associated with chronic thrombotic conditions or disorders, chronic CD40 ligand-mediated conditions or disorders, or chronic thromboxane-mediated conditions or disorders.

Gene therapy approaches for treating these conditions utilize an expression vector or plasmid that contains therein a recombinant gene encoding an inducer of a PPARγ agonist. The recombinant gene can be introduced, using the expression vector, into one or more target tissues or systemically to achieve subsequent expression of the inducer of a PPARγ agonist (either constitutively, inducibly, or in a tissue specific manner). The recombinant gene includes, operatively coupled to one another, an upstream promoter operable in mammalian cells, and other suitable regulatory elements (i.e., enhancer or inducer elements), a coding sequence that encodes the inducer of a PPARγ agonist, and a downstream transcription termination region. Any suitable constitutive promoter or inducible promoter can be used to regulate transcription of the recombinant gene, and one of skill in the art can readily select and utilize such promoters, whether now known or hereafter developed. Known recombinant techniques can be utilized to prepare the recombinant gene, transfer it into the expression vector, and administer the vector to a patient. Exemplary procedures are described in Sambrook et al., Molecular Cloning: A Laboratory Manual, Second Edition, Cold Spring Harbor Press, NY (1989), which is hereby incorporated by reference in its entirety. One of skill in the art can readily modify these procedures, as desired, using known variations of the procedures described therein.

With respect to the construction of viral vectors for delivery of either (i) a DNA molecule encoding an inducer of a PPARγ agonist such as decorin, or (ii) a DNA molecule encoding an enzyme that catalyzes formation of prostaglandin D₂ precursor, such as PGD synthases, or (iii) a DNA molecule encoding PPARγ, can be used.

Beta-trace (or lipocalin form or brain form) of PGD synthases and its cDNA are disclosed in White et al., “Structure and Chromosomal Localization of the Human Gene for a Brain Form of Prostaglandin D2 Synthase,” J. Biol. Chem. 267(32):23202-23208 (1992); and Genbank Accession Nos. NM_{—}000954, AH005144 (promoter and exon 1), M98537 (promoter and exon 1), M61901, and M98538 (exons 2-6), each of which is hereby incorporated by reference in its entirety. Although the above-referenced sequences relate to human PGD synthase (brain form), sequences are also known for other mammals such as rat and mouse.

The glutathione-dependent (or hematopoietic form) of PGD synthases and its cDNA are disclosed in Kanaoka et al., “Structure and Chromosomal Localization of Human and Mouse Genes for Hematopoietic Prostaglandin D Synthase: Conservation of the Ancestral Genomic Structure of Sigma-Class Glutathione S-Transferase,” Eur. J. Biochem. 267: 3315-3322 (2000); and Genbank Accession Nos. D82073, NM_{—}014485, and AB008825-AB008830 (exons 1-6), each of which is hereby incorporated by reference in its entirety.

The human decorin protein and cDNA are disclosed in Vetter et al., “Human Decorin Gene: Intron-Exon Junctions and Chromosomal Localization,” Genomics 15:161-168 (1993); and Genbank Accession Nos. AH002681 and L01125-L01131, each of which is hereby incorporated by reference in its entirety. Fragments of decorin, including known isoforms thereof, can likewise be utilized in accordance with the present invention. A number of such isoforms of human decorin have previously been identified and reported in Genbank. Although the above-referenced sequences relate to human decorin, sequences are also known for other mammals such as rat.

The human PPARγ protein and its encoding cDNA are disclosed in Greene et al., “Isolation of the Human Peroxisome Proliferator Activated Receptor Gamma cDNA: Expression in Hematopoietic Cells and Chromosomal Mapping,” Gene Expr. 4(4-5):281-299 (1995); Elbrecht et al., “Molecular Cloning, Expression and Characterization of Human Peroxisome Proliferator Activated Receptors Gamma 1 and Gamma 2,” Biochem. Biophys. Res. Commun. 224(2):431-437 (1996); and Genbank Accession Nos. NM_{—}138712, NM_{—}005037, and NM_{—}015869, each of which is hereby incorporated by reference in its entirety. Multiple transcript variants that use alternate promoters and splicing have been identified for PPARγ. At least three of these variants encode the same isoform.

Delivery of the expression vector or naked plasmid DNA to patient cells that are intended to be transformed can be carried out according to known procedures, which includes delivery of a composition containing a high titer of the infective transformation system or naked plasmid into the site where targeted cells reside. The composition can be provided as a single administration, multiple administration, or in the form of a sustained-release DNA delivery vehicle. The targeted cells/tissues include, generally and without limitation, vascular tissues, bone marrow, and structural cells. As a result, the likelihood of infecting the desired or targeted cells is significantly increased over non-targeted systemic administration.

When transforming mammalian cells for heterologous expression of a protein or polypeptide, a viral vector or naked (plasmid) DNA can be employed.

Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. Adeno-associated viral gene delivery vehicles can be constructed and used to deliver a gene to cells. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l. Acad. Sci. USA 90:10613-10617 (1993); and Kaplitt et al., Nature Genet. 8:148-153 (1994), each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; and U.S. Pat. No. 5,885,808 to Spooner et al.; and U.S. Pat. No. 5,871,727 to Curiel, each of which is hereby incorporated by reference in its entirety).

Retroviral vectors which have been modified to form infective transformation systems can also be used to deliver nucleic acid encoding a desired protein or polypeptide or RNA product into a target cell. One such type of retroviral vector is disclosed in U.S. Pat. No. 5,849,586 to Kriegler et al., which is hereby incorporated by reference in its entirety.

Liposomal delivery systems can be used to deliver expression vectors or plasmid DNA into targeted cells. Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Nat'l Acad. Sci. USA 84:7851 (1987); Biochemistry 28:908 (1989), which is hereby incorporated by reference). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

DNA delivery vehicles in the form of a sustained-release polymeric matrix containing the DNA to be delivered can likewise be employed to administer the DNA for purposes of gene therapy.

In addition to the foregoing, it is contemplated that administration of the above-identified agents in accordance with the present invention will modulate megakaryocytes. That is, by increasing the concentration of PPARγ present in the megakaryocytes, the megakaryocytes will be altered phenotypically. As a consequence of such treatment, it is believed that daughter platelets produced by those megakaryocytes will likewise differ phenotypically from platelets produced prior to administration of the above-identified agents. Without being bound by belief, it is believed that the platelets can differ in their composition (e.g., more PPARγ would lead to platelet dampening). Such daughter platelets may also have altered expression of CD40 ligand and/or cyclooxygenase 1, which if diminished would blunt or minimize pro-inflammatory and/or pro-thrombotic responses by the platelets. The platelets would be characterized by a diminished ability to activate, aggregate, and form clots.

The present invention also relates to several diagnostic assays.

One such assay relates to a method of assessing the activity of a compound as a PPARγ agonist. This assay can be carried out by combining the compound with both thrombin and platelets, determining the level of CD40 ligand or thromboxane released from the platelets, and comparing the level of CD40 ligand or thromboxane released from the platelets to the level of CD40 ligand or thromboxane released from a standard, wherein deviation from the standard, or absence thereof, indicates activity of the compound as a PPARγ agonist.

In the absence of PPARγ agonist activity, a platelet activator (e.g., thrombin, epinephrin, collagen, ADP, etc.) will contribute to platelet activation and release of CD40 ligand and/or thromboxanes; however, with such activity, the induced activation will be blunted and the amount of CD40 ligand or thromboxane released will be reduced. Thus, by comparison to the standard, an assessment can be made as to whether the compound has PPARγ agonist activity. In accordance with one embodiment, the standard includes platelets in the absence of the platelet activator, and the comparison assesses the absence of deviation between the combination and the standard. In accordance with another embodiment, the standard includes platelets, the platelet activator, and a known PPARγ agonist, and the comparison assesses the deviation between the combination and the standard.

Regardless of the standard selected, the assay preferably utilizes an immunological detection procedure, using an antibody or binding portion thereof recognizing CD40 ligand or thromboxane B₂. The sample (and the standard) is contacted with the antibody or binding portion thereof and any reaction which indicates that CD40 ligand or thromboxane B₂ is present in the sample is detected. Detection of antibody-CD40 ligand or antibody-thromboxane B₂ binding can be achieved using any of a variety of known detection procedures, such as enzyme-linked immunoabsorbent assay, radioimmunoassay, gel diffusion precipitin reaction assay, immunodiffusion assay, agglutination assay, fluorescent immunoassay, protein A immunoassay, immunoelectrophoresis assay, Western blot, immunodotblot assay, or immunoslotblot assay.

Suitable anti-CD40 ligand antibodies useful for these procedures include polyclonal antibodies and monoclonal antibodies. Exemplary monoclonal antibodies include, without limitation, MK13 (Boehringer Ingelheim), 24-31 (Ancell), TRAP1 (Calbiochem Corp.), and Clone C20 (Santa Cruz). Other anti-CD40 ligand antibodies or binding fragments thereof can also be used.

Suitable anti-thromboxane (A₂ or B₂) antibodies useful for these procedures include polyclonal antibodies and monoclonal antibodies. Exemplary polyclonal antibodies include, without limitation, rabbit anti-TXB₂ (Novus Biologicals, Littleton, Colo.; Cayman Chem. Co., Ann Arbor, Mich.).

Another assay of the present invention relates to a method of diagnosing a CD40 ligand- or thromboxane-mediated condition through the use of a patient sample. Suitable patient sample materials include, without limitation, blood, plasma, tissue washings, lung lavage, eye fluids, saliva, joint fluid, peritoneal fluid, stool, semen, gastric fluids, and thoracic fluids. Having thus obtained a sample, the level of PPARγ in the patient sample is detected, wherein a reduced PPARγ level indicates the presence of a CD40 ligand- or thromboxane-mediated condition (such as those listed above).

The assay preferably utilizes an immunological detection procedure, using an antibody or binding portion thereof recognizing PPARγ. The sample is contacted with the antibody or binding portion thereof and any reaction which indicates that PPARγ is present in the sample is detected. Detection of antibody-PPARγ binding can be achieved using any of a variety of known detection procedures, such as enzyme-linked immunoabsorbent assay, radioimmunoassay, gel diffusion precipitin reaction assay, immunodiffusion assay, agglutination assay, fluorescent immunoassay, protein A immunoassay, immunoelectrophoresis assay, Western blot, immunodotblot assay, or immunoslotblot assay.

Suitable anti-PPARγ antibodies useful for these procedures include polyclonal antibodies and monoclonal antibodies. Exemplary monoclonal antibodies include, without limitation, the monoclonal anti-PPARγ antibody from Santa Cruz Biotechnology Inc., (Santa Cruz, Calif.). Exemplary polyclonal antibodies include, without limitation, the polyclonal anti-PPARγ antiserum available from Calbiochem® Immunochemicals/EMD Biosciences (San Diego, Calif.).

A further assay of the present invention relates to a method of assessing the efficacy of a PPARγ agonist therapy through the use of a sample from a patient who has previously received/been administered a PPARγ agonist or an inducer of a PPARγ agonist for treating a medical condition or disorder. Suitable patient sample materials include, without limitation, blood, plasma, tissue washings, lung lavage, eye fluids, saliva, joint fluid, peritoneal fluid, stool, semen, gastric fluids, and thoracic fluids. Having thus obtained a sample, the level of PPARγ in the patient sample is detected and determined, wherein an elevated PPARγ level, relative to a baseline PPARγ level for the patient prior to the administration of the agonist or inducer, indicates the efficacy of the PPARγ agonist therapy.

The assay preferably utilizes an immunological detection procedure, using an antibody or binding portion thereof recognizing PPARγ. The sample is contacted with the antibody or binding portion thereof and any reaction which indicates that PPARγ is present in the sample is detected. Detection of antibody-PPARγ binding can be achieved using any of the above-identified immunoassay, with any of the anti-PPARγ antibodies described above as reagents.

### EXAMPLES

The following examples are intended to illustrate, but by no means are intended to limit, the scope of the present invention as set forth in the appended claims.

Materials and Methods

Cell Line and Culture Conditions

Meg-01 cells were purchased from the American Type Culture Collection (Rockville, Md.) and are widely used as a model of human megakaryocytes (Ogura et al., “Establishment of a Novel Human Megakaryoblastic Leukemia Cell Line, MEG-01, with Positive Philadelphia Chromosome,” Blood 66:1384-1392 (1985), which is hereby incorporated by reference in its entirety). Meg-01 cells were cultured in RPMI-1640 tissue culture medium (Invitrogen, Carlsbad, Calif.) supplemented with 10% fetal bovine serum (FBS) (Invitrogen), 10 mM HEPES (Sigma, St. Louis, Mo.), 2 mM L-glutamine (Invitrogen), 4.5 g/L glucose (Invitrogen) and 50 μg/ml gentamicin (Invitrogen).

Preparation of Platelets

Blood samples (500 mL) were collected from healthy volunteers by venipuncture into a CPDA-1 blood collection bag (Baxter Healthcare, Dearfield, Ill.). The platelet-rich plasma was obtained by centrifugation at 1,800×g for 8 minutes and extracted into the transfer bag (Charter Medical, Winston-Salem, N.C.) at room temperature. The Pall Biomedical Purecell LRF high efficiency leukoreduction filter was used to reduce leukocytes, microaggregates and anaphylatoxin C3a. Leukocytes were removed by adherence in the filter. Platelets were washed with 0.9% saline using a COBE 2991 Blood Cell Processor (Lakewood, Colo.). Cell counts were performed on an Abbott Cell-Dyn 1700 (Abbott Park, Ill.) and the final platelet count was 5.5×10¹⁰/unit. The maximum numbers of contaminant non-platelet cells were 1×10⁵ white blood cells and 1×10⁸ red blood cells, the percentages being 0.0001% and 0.1818% of platelets, respectively. Pooled platelet rich plasma was prepared by the same procedure from 2-5 donors and combined into a pool bag (Charter Medical). The platelets were isolated by additional centrifugation step at 1,200×g of the platelet rich plasma for 4 minutes and the pellet was washed twice with 1× PBS.

Western Blot for PPARγ

Meg-01 and platelet total protein was isolated using Nonidet P-40 lysis buffer containing a protease inhibitor cocktail (4-(2-aminoethyl)-benzenesulfonyl fluoride, pepstatin A, transepoxysuccinyl-L-leucylamido (4-guanidino) butane, bestatin, leupeptin and aprotinin) (Sigma). Total protein was quantified with a BCA protein assay kit (Pierce, Rockford, Ill.). A total of 15 μg of protein was electrophoresed on 10% denaturing polyacrylamide-stacking gels and transferred to nitrocellulose membrane (Amersham, Piscataway, N.J.) at 4° C. After blocking with 10% Blotto (PBS/0.1% Tween 20 and 10% milk) for 2 hours at room temperature, membranes were then incubated with a mouse monoclonal anti-PPARγ antibody from Santa Cruz Biotechnology (1:1000) (Santa Cruz, Calif.) or with a rabbit polyclonal anti-PPARγ antibody from Calbiochem (1:5000) (San Diego, Calif.) diluted in 2.5% Blotto for 1 hour. They were then washed in PBS/0.1% Tween 20 and incubated with a goat anti-rabbit-HRP (Santa Cruz) secondary antibody at 1:2000 dilution for 1 hour. The membranes were washed in PBS/0.1% Tween 20 and bands were visualized using a Western Lightning chemiluminescence kit according to the manufacturer's instructions (Perkin Elmer Life Sciences, Boston, Mass.). The platelet PPARγ band detected by Western Blot was identified as PPARγ by MALDI-TOF Mass Spectroscopy (MS) peptide mapping analysis at the University of Rochester Micro Chemical Protein/Peptide Core Facility.

Meg-01 and Human Platelet Immunocytochemistry for PPARγ

One×10⁵ Meg-01 cells and 1×10⁷ platelets were cytospun on slides and fixed with 1% paraformaldehyde and stained with a rabbit polyclonal anti-PPARγ antibody (Santa Cruz) or with an IgG isotype control (both at 4 μg/ml) (Santa Cruz) as described (Harris and Phipps, “rostaglandin D₂, its Metabolite 15-d-PGJ₂, and Peroxisome Proliferator Activated Receptor-gamma Agonists Induce Apoptosis in Transformed, but not Normal, Human T Lineage Cells,” Immunology 105:23-34 (2002), which is hereby incorporated by reference in its entirety). Slides were developed with AEC reagent (Zymed Laboratories, San Francisco, Calif.) and visualized with an Olympus BX51 microscope. Photographs were taken using a SPOT camera with SPOT RT software (New Hyde Park, N.Y.).

Preparation of Human Bone Marrow Smears and Immunocytochemistry for PPARγ

Human bone marrow aspiration material was obtained from the hip bone of anemia patients. A drop of material about 2 mm in diameter was put onto slides and immediately spread over by cover slip and air dried for 24 hours. Smears were fixed with acetone-methanol solutions. Except for the fixation step, immunocytochemistry was performed as described (Harris and Phipps, “Prostaglandin D₂, its Metabolite 15-d-PGJ₂, and Peroxisome Proliferator Activated Receptor-gamma Agonists Induce Apoptosis in Transformed, but not Normal, Human T Lineage Cells,” Immunology 105:23-34 (2002), which is hereby incorporated by reference in its entirety). Slides were stained with a mouse monoclonal anti-PPARγ antibody (Santa Cruz) or with IgG1 isotype control (both at 4 μg/ml) (Santa Cruz) and biotin-labeled horse anti-mouse IgG (Vector Laboratories, Burlingame, Calif.) was used as secondary antibody. After staining for PPARγ, counterstaining with hematoxylin was performed. One slide from the same patient was stained with a Diff-Quik stain set (Dade Behring, Newark, Del.).

cDNA Synthesis and RT-PCR Assay

Total RNA was extracted with Tri-Reagent from platelets and Meg-01 according to the supplier's protocol (MRC, Cincinnati, Ohio). A total of 2 μg of RNA was used for the reverse transcription reaction and RT-PCR for PPARγ and β-actin was performed as described (Harris and Phipps, “Prostaglandin D₂, its Metabolite 15-d-PGJ₂, and Peroxisome Proliferator Activated Receptor-gamma Agonists Induce Apoptosis in Transformed, but not Normal, Human T Lineage Cells,” Immunology 105:23-34 (2002), which is hereby incorporated by reference in its entirety). A reaction was performed without reverse transcriptase for each cDNA synthesis and used as a negative control in the PCR. Ten μl of cDNA was used in the PCR reaction. The RT-PCR products were separated by gel electrophoresis on 1% agarose gels and stained with ethidium bromide. Adipose tissue and THP1 human monocyte cells were used as positive controls.

Flow Cytometric Analysis

The washed platelets were re-suspended and incubated in 1 ml FACS lysis solution (FLS, BD Biosciences, Immunocytometry Systems, San Jose, Calif.) at a concentration of 1×10⁷/ml in 1×FLS for 10 minutes in the dark at room temperature. After centrifugation at 500×g for 5 minutes, the cells were permeabilized with 1× FLS+0.2% saponin (Sigma) for 10 minutes. Samples then were incubated with 8 μg/ml monoclonal FITC-labeled anti-PPARγ antibody (BD Biosciences, San Diego, Calif.) or FITC-labeled IgG1 isotype control (BD Biosciences) for 30 minutes in the dark at room temperature. Cells were washed with 1×PBS containing 1% bovine serum albumin (BSA) and 0.1% sodium azide (NaN₃). Samples were re-suspended in 1% PFA and analyzed on a Becton Dickinson FACS Calibar flow cytometer.

For CD40L surface staining, washed platelets were pre-treated with PPARγ agonists for 15 minutes and were then exposed to 0.8 U/ml thrombin for 60 min at 37° C. in the presence of 200 μM fibrinogen receptor antagonist (Bachem, King of Prussia, Pa.) and 5 mM EDTA (Sigma) to prevent clotting. The platelets were then stained for CD40L using a mouse IgG1 anti-human CD40L biotinylated monoclonal antibody (Ancell, Bayport, Minn.), or a mouse IgG1 isotype control antibody (Caltag, Burlingame, Calif.) followed by streptavidin conjugated to allophycocyanin (Caltag).

PPARγ Activity Assay

Concentrated platelets were washed twice and treated with 20 μM 15d-PGJ₂ (Biomol, Plymouth Meeting, Pa.), rosiglitazone (Cayman Chemical, Ann Arbor, Mich.), ciglitazone (Biomol), or DMSO (vehicle control) for 2 hours at 37° C. Platelets were lysed with hypotonic buffer (10 mM HEPES-KOH (pH 7.9), 1.5 mM MgCl₂, 10 mM KCl, 0.5 mM DTT, 0.5% Nonidet P-40 and 0.2 mM PMSF) and 10 μg of cell extract was incubated in each well of TransAM® PPARγ assay kit (Active Motif, Carlsbad, Calif.) and PPARγ DNA binding was determined as per manufacturers' protocol.

Electrophoretic Mobility Shift Assay for PPARγ

Nuclear extracts of Meg-01 cells were prepared as described previously (Andrews and Faller, “A Rapid Micropreparation Technique for Extraction of DNA Binding Proteins from Limiting Numbers of Mammalian Cells,” Nucleic Acids Res. 19:2499 (1991), which is hereby incorporated by reference in its entirety). Cells were treated with 5 μM 15d-PGJ₂, 10 μM ciglitazone, or DMSO (vehicle control) for 4 hours. The cells were washed in cold PBS and then incubated on ice in hypotonic buffer (10 mM HEPES-KOH (pH 7.9), 1.5 mM MgCl₂, 10 mM KCl, 0.5 mM DTT, 0.5% Nonidet P-40 and 0.2 mM PMSF) for 10 minutes. The lysates were vortexed for 10 seconds and centrifuged for 15 seconds. The pellet was isolated carefully and re-suspended in 80 μl of hypertonic buffer (20 mM HEPES-KOH (pH 7.9), 1.5 mM MgCl₂, 25% glycerol, 420 mM NaCl, 0.2 mM EDTA, 0.5 mM DTT, and 0.2 mM PMSF). After incubation on ice for 20 minutes, lysates were centrifuged for 20 seconds and the supernatant containing the nuclear protein was transferred to new tubes. Protein quantification was performed using a BCA assay kit. Platelet protein isolation was done as described for the PPARγ activity assay. For the gel shift assay of Meg-01 and platelets, the consensus sequence for PPARγ (5′-CAAAACTAGGTCAAAGGTCA-3′) (SEQ ID NO: 1) was labeled with [γ-32P]ATP using T4 Polynucleotide Kinase (Life technologies). Micro Bio-Spin P-30 Tris Chromatography Columns were used to remove the unbound nucleotides (Bio-Rad). Meg-01 or platelet protein extracts were incubated with binding buffer (10 mM Tris-HCl (pH 7.5), 50 mM NaCl, 4% glycerol, 1 mM MgCl₂, 0.5 mM EDTA, 0.5 mM DTT and 0.05 mg/ml poly (dI-dC)) and 50,000 counts of labeled oligonucleotide or cold oligonucleotide for 15 minutes at room temperature. Supershift experiments were completed by adding 2 μg of the anti-PPARγ antibody (Calbiochem) to the binding reaction. The samples were then run on a 4% non-denaturing polyacrylamide gel. The gel was dried on a Savant SGD 2000 gel dryer (Savant, Farmingdale, N.Y.) for 1 hour at 50° C., and exposed to film overnight.

Measurement of CD40L and TXB₂

Platelets were isolated as described above and cultured with buffer or with 15d-PGJ₂ or rosiglitazone (both at 20 μM) for 15 minutes at 37° C. Platelets were then activated with 0.8 U/ml thrombin or buffer and samples were taken at the 5, 10, 15, 30 and 60 minutes time points to measure human soluble CD40L and PGE₂. CD40L assays were performed with a commercially available ELISA specific for CD40L (Bender Biomedical Systems, San Bruno, Calif.). Virtually identical results were obtained using an ELISA for CD40L developed in our lab (data not shown). The stable end product of platelet TXA₂ synthesis, namely TXB₂, was measured using a highly specific enzyme immunoassay from Cayman Chemical Company as per the manufacturer's directions.

Platelet Aggregation and ATP Release

Platelet aggregation was performed using the turbidometric method of Born (“Quantitative Investigations into the Aggregation of Blood Platelets,” J. Physiol. Lond. 162:67 (1962), which is hereby incorporated by reference in its entirety) with simultaneous measurement of ATP release using a Chrono-log Lumi-aggregometer with AGGRO/LINK for Windows Software version 5.1.6 (Chrono-log Corp., Havertown, Pa.). Blood was collected by clean venipuncture from normal donors who abstained from drugs known to affect platelet aggregation into 0.105M/sodium citrate. Platelet rich plasma (PRP) was prepared by centrifugation at 150×g for 10 min at 20° C., and the platelet count adjusted to 250,000/μl by mixing PRP and platelet poor plasma from the same donor. All experiments were performed within 3 hours of blood collection. Aggregation was performed with ADP, and the slope of aggregation and amplitude were computed using accompanying software. The effects of the PPARγ agonist 15d-PGJ₂ were tested by addition of varying concentrations to PRP for 15 min before aggregation. The 15d-PGJ₂ was dissolved in DMSO, with a final concentration of DMSO in the samples of approximately 0.1%. Control experiments showed no effect of this concentration of DMSO on platelet aggregation or release. Additional clotting studies were performed using a PFA-100 according to the manufacturer's instructions. Basically, clotting time (induced by clotting activator such as epinephrine or ADP) was measured in the presence or absence of a PPARγ agonist, and differences noted.

Statistics

Statistical analysis of time dependent changes in supernatant levels of sCD40L and TXB₂ employed the log rank test performed using Statview (SAS Institute, Cary, N.C.). P values of <0.05 were considered significant.

### Example 1

### Meg-01 Megakaryocytes and Human Blood Platelets Express PPARγ Protein

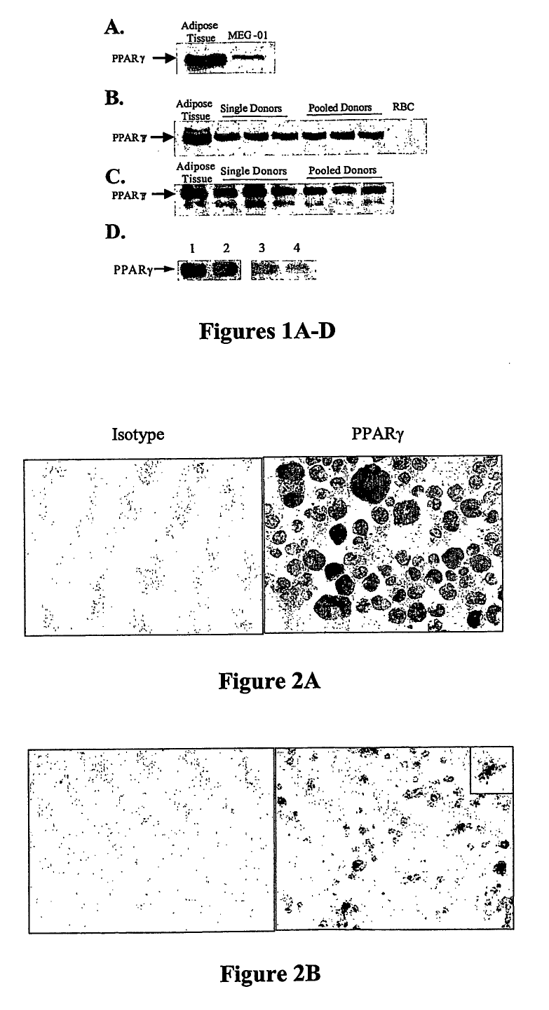

Meg-01 cells have been extensively used as a model of human megakaryocytes (Ogura et al., “Establishment of a Novel Human Megakaryoblastic Leukemia Cell Line, MEG-01, with Positive Philadelphia Chromosome,” Blood 66:1384-1392 (1985), which is hereby incorporated by reference in its entirety). To determine whether megakaryocytes and platelets express PPARγ protein, Meg-01 cells and human platelets were tested by western blot for PPARγ. Meg-01 cells and platelets were lysed and the protein analyzed for PPARγ by western blot using commercially available and widely used anti-PPARγ antibodies. Meg-01 cells express PPARγ protein that co-migrated with human fat tissue PPARγ, used as a known positive control (FIG. 1A). We next evaluated highly purified human platelets for PPARγ expression. Three different single donor platelets and three multiple donor pooled platelet samples were tested for PPARγ using two different anti-PPARγ antibodies (FIGS. 1B and 1C). Human platelets express a PPARγ band, which migrated similarly to the adipose tissue PPARγ band. While the platelet preparations were highly purified (>99.99% platelets), they did contain the rare white blood cell. To determine how many white blood cells were needed to generate a PPARγ band on a western blot, experiments were completed with different numbers of white blood cells. At least 1×10⁶ white blood cells were needed to show a PPARγ band on western blots. Therefore contamination with white blood cells in purified platelets could not account for the western blot signal. Western blot experiments of red blood cells were also were completed for PPARγ and red blood cells do not express PPARγ (see FIG. 1B). Additionally, PPARγ of platelet origin was confirmed by MALDI-TOF Mass Spectroscopy peptide mapping.

To determine whether other species platelets express PPARγ, purified mouse platelets were similarly tested. As with the human platelets, the mouse platelets were also found to express PPARγ protein (FIG. 1D).

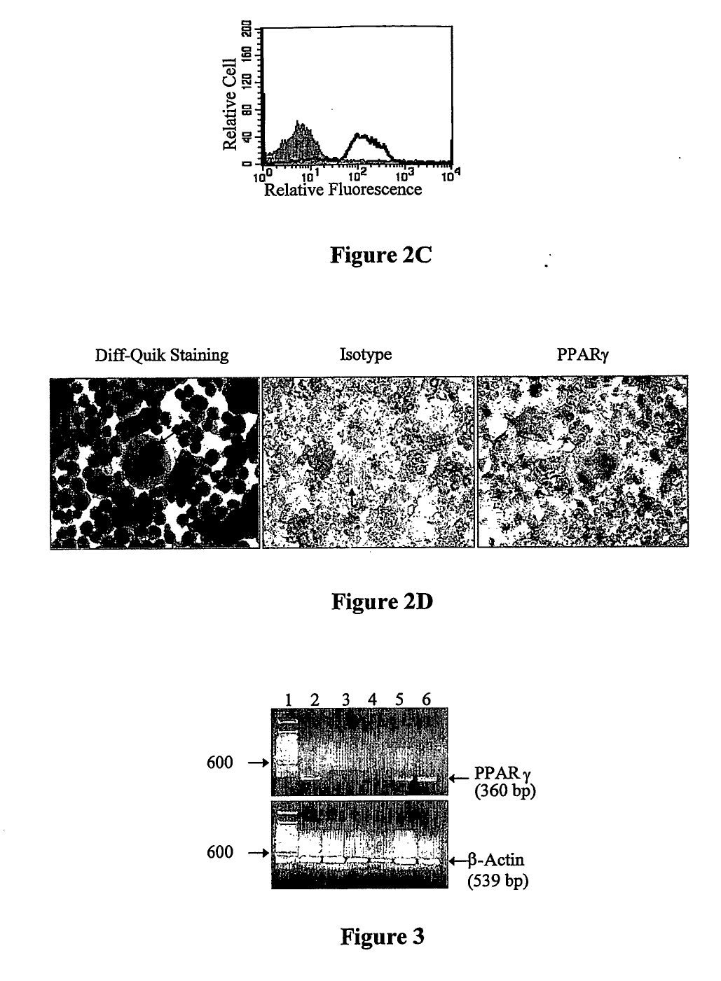

The presence of PPARγ in Meg-01 cells and human platelets was further examined by immunocytochemistry. Meg-01 cells (FIG. 2A) and platelets (FIG. 2B) contain PPARγ protein, confirming the western blot data. The PPARγ staining pattern of Meg-01 is cytoplasmic, as well as nuclear. In platelets, the staining pattern for PPARγ appeared throughout the cell, with apparent denser staining in platelet granules.

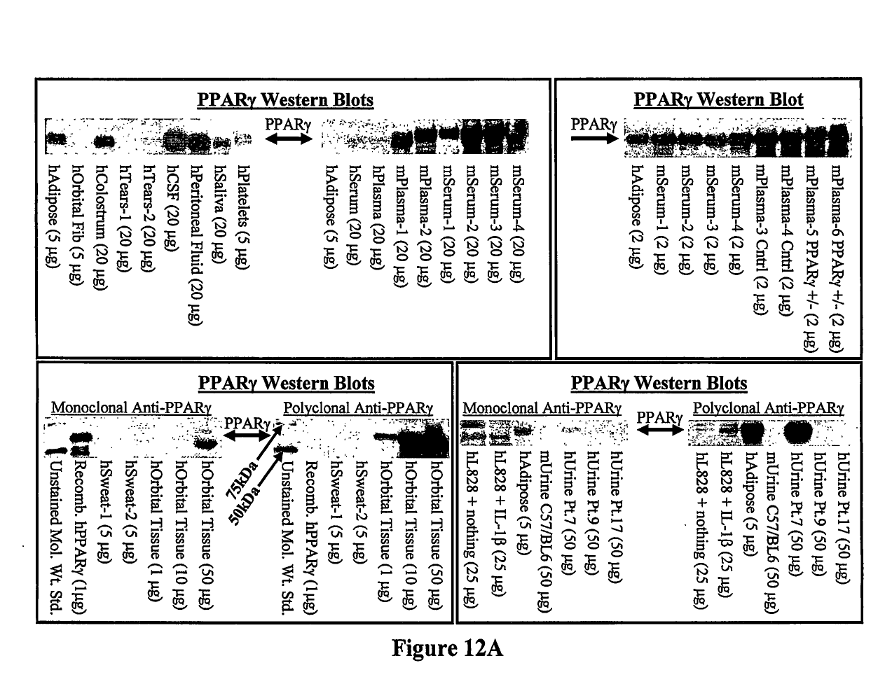

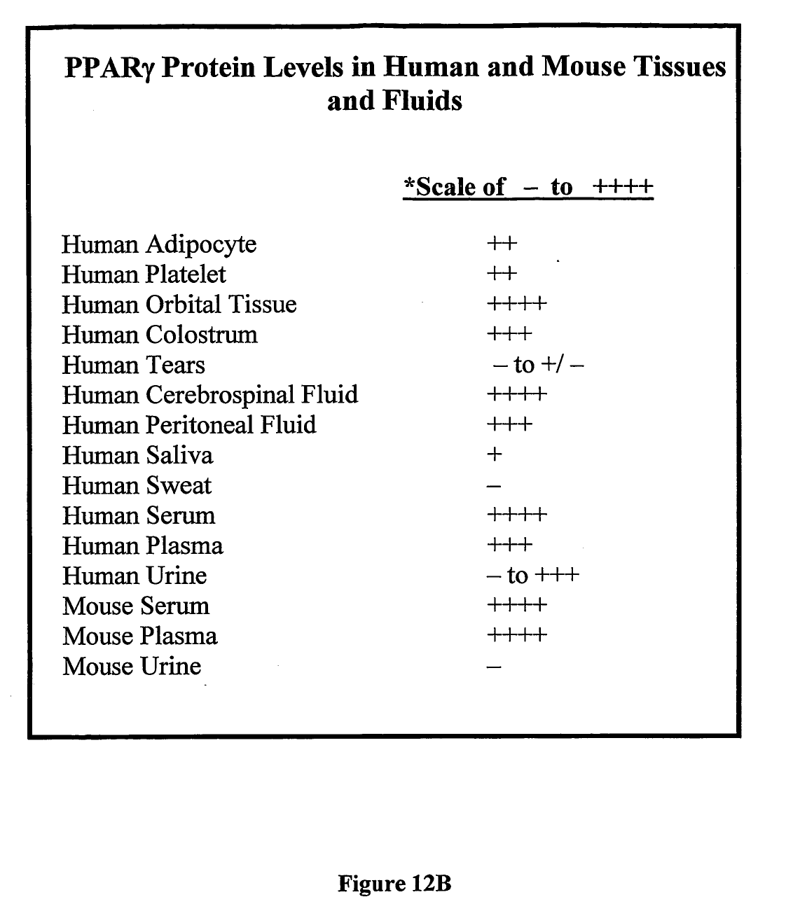

To further demonstrate expression of PPARγ protein in human platelets, flow cytometry experiments were performed. Concentrated and washed human platelets were incubated with monoclonal FITC-labeled anti-PPARγ antibody or FITC-labeled IgG1 isotype for 30 minutes and analyzed on a Becton Dickinson FACS Caliber flow cytometer. Platelets, being very small enucleate cells, have a low forward and side-scatter profile compared with white blood cells. The flow cytometry results showed that PPARγ protein was expressed in more than 85% of platelets (FIG. 2C). As shown in FIGS. 12A-B, PPARγ is present in detectable amounts in adipocyte, platelets, orbital tissue, colostrum, cerebral spinal fluid, peritoneal fluid, saliva, serum, plasma, and urine.

### Example 2

### Human Bone Marrow Megakaryocytes Express PPARγ Protein

Based on the fact that platelets and the Meg-01 cells expressed PPARγ protein, it was expected that human megakaryocytes would also express PPARγ protein. Expression of PPARγ in human bone marrow megakaryocytes was detected by immunocytochemistry using a monoclonal anti-PPARγ antibody. Human bone marrow was stained with Diff-Quik to identify human megakaryocytes (FIG. 2D). The megakaryocyte is the largest cell of bone marrow with multi-lobated nuclei and abundant granular cytoplasm. B one marrow smears were also prepared for immunocytochemistry to stain for PPARγ. The right-hand panel of FIG. 2D shows staining of human megakaryocytes for PPARγ. The middle panel shows no staining with an isotype control antibody (smear is lightly counterstained with hematoxylin).

### Example 3

### PPARγ mRNA is Expressed in the Meg-01 Cell Line but not in Platelets

Expression of PPARγ mRNA in Meg-01 and platelets was examined by RT-PCR. Platelets, while enucleate, do express a range of mRNA species (Gnatenko et al., “Transcript Profiling of Human Platelets Using Microarray and Serial Analysis of Gene Expression,” Blood 101:2285-2293 (2003), which is hereby incorporated by reference in its entirety). Total RNA was isolated from Meg-01 cells and single donor or pooled platelets, and then reverse transcribed as described in the Materials and Methods section. Resulting cDNA was run in PCR reactions with control β-actin primers or primers specific for human PPARγ. RNA from human adipose tissue and THP1 human monocyte cells was used as positive controls for PPARγ. The results revealed a single RT-PCR product of the expected size of 360 bp for PPARγ in adipose tissue (FIG. 3, lane 2 ). Meg-01 cells and the THP-1 monocytic cells express PPARγ mRNA (FIG. 3, lanes 6 and 7, respectively). PPARγ mRNA was not present in platelet samples (FIG. 3, lanes 3-5). All samples did express β-actin mRNA, consistent with reports that platelets express mRNA encoding β-actin (Inwald et al., “CD40 is Constitutively Expressed on Platelets and Provides a Novel Mechanism for Platelet Activation,” Circ. Res. 92:1041-1048 (2003), which is hereby incorporated by reference in its entirety).

### Example 4

### Meg-01 PPARγ has DNA Binding Ability that is Enhanced by Treatment with PPARγ Ligands

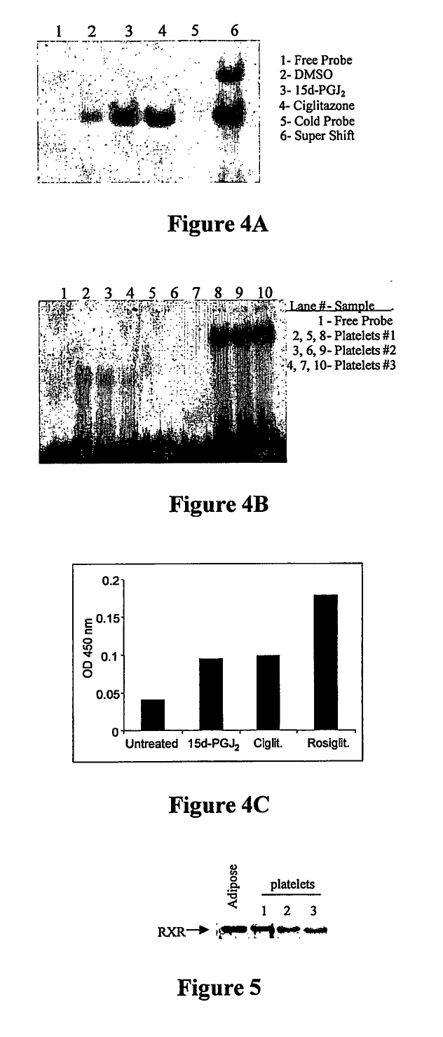

To determine if the PPARγ protein in Meg-01 cells can bind DNA, gel shift assays were performed. In many systems enhanced DNA binding is observed if PPARγ -expressing cells are first exposed to a PPARγ agonist (Juge-Aubry et al., “DNA Binding Properties of Peroxisome Proliferator-Activated Receptor Subtypes on Various Natural Peroxisome Proliferator Response Elements,” J. Biol. Chem. 272:25252-25259 (1997), which is hereby incorporated by reference in its entirety). Meg-01 cells were treated with the PPARγ agonists 15d-PGJ₂ (5 μM) or ciglitazone (10 μM) or vehicle (DMSO) for 4 hours in culture. Nuclear protein was then incubated with a radio-labeled probe containing the consensus DNA binding sequence for PPARγ (FIG. 4A). FIG. 4A shows that Meg-01 cells have a constitutive level of active PPARγ (lane 2 ), which was increased by exposure to the natural PPARγ agonist 15d-PGJ₂ (lane 3) and to the synthetic PPARγ agonist ciglitazone (lane 4). A supershift using an anti-PPARγ antibody further supported PPARγ expression in Meg-01 cells (lane 6). 15d-PGJ₂ and ciglitazone increase the activation of PPARγ in Meg-01 cells.

### Example 5

### Platelets have Constitutively Active PPARγ Protein that has DNA Binding Ability

EMSA was next performed to determine if platelet PPARγ protein can bind to the DNA PPAR response element. Lysates from three different rigorously purified platelet samples were incubated with a radioactive probe (PPARγ consensus DNA binding sequence) or cold probe (FIG. 4B). A discrete DNA binding band appears in the three different platelet samples (lanes 2-4). The band disappears when extracts were incubated with excess cold probe (lanes 5-7a). A supershift assay using a specific anti-PPARγ antibody was also performed and the bands shifted to a higher mass consistent with PPARγ. (lanes 8-10). The ability of platelet derived PPARγ to bind its DNA consensus sequence was also measured using the TransAM™ PPARγ assay kit (Active Motif Inc.). In this method the consensus DNA sequence for PPARγ binding (or as a control mutated oligonucleotides) is plate-bound. A cell lysate is then added to the well, washed and next incubated with an enzyme-conjugated anti-PPARγ antibody that recognizes only DNA-bound PPARγ. Following substrate addition, a colored product is formed. Platelets were exposed to buffer, 15d-PGJ₂, ciglitazone, or rosiglitazone (20 μM for all) for 2 h at 37° C. and then protein extracted. The measurements demonstrate that platelet PPARγ binds DNA even without treatment with PPARγ agonists, but bind 3-4 fold more strongly in the presence of PPARγ agonists (FIG. 4C). The ability of platelet PPARγ to bind DNA in the absence of deliberate addition of PPARγ ligand suggests that platelets do contain an endogenous ligand. One possible ligand is lysophosphatidic acid, which platelets are known to produce (McIntyre et al., “Identification of an Intracellular Receptor for Lysophosphatidic Acid (LPA): LPA is a Transcellular PPAR Gamma Agonist,” Proc. Natl. Acad. Sci. USA 100:131-136 (2003), which is hereby incorporated by reference in its entirety). Overall, these results further support that platelets express PPARγ and that platelet PPARγ retains its DNA binding ability.

### Example 6

### PPARγ Agonists Prevent Activated Platelet Release of CD40L, TXB₂, PGE₂, and ATP and Inhibit Platelet Aggregation

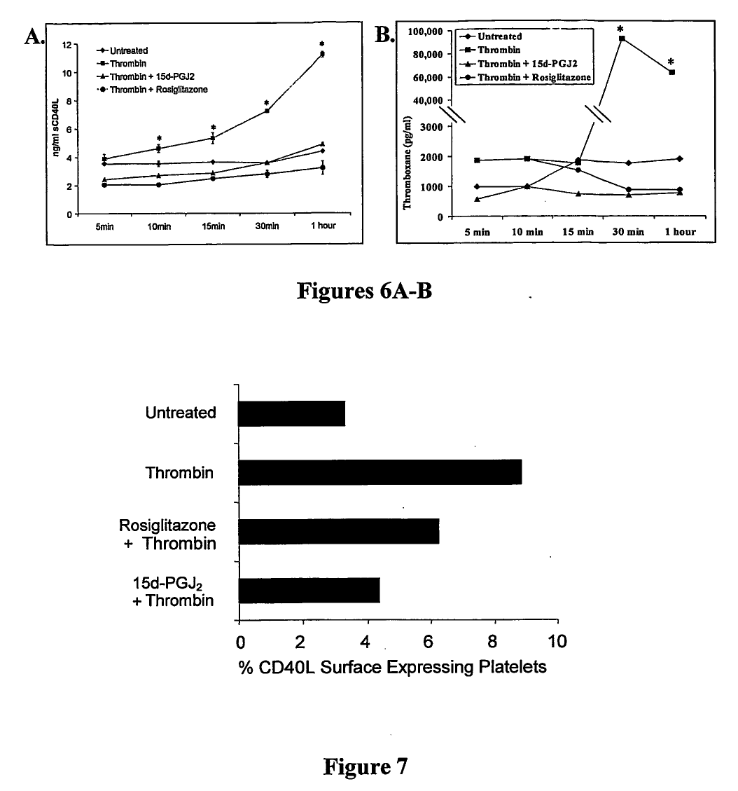

From the foregoing, it was suspected that platelet PPARγ played a role in attenuating platelet activation. To test the theory, human platelets were isolated and exposed to the PPARγ ligands 15d-PGJ₂ or rosiglitazone for 15 min at 37° C. Platelets were then incubated with buffer or with thrombin, a powerful platelet activator. Upon platelet activation, the cells expel key bioactive mediators important for thrombosis, inflammation and vascular disease including CD40L, TXB₂, and PGE₂ (Phipps et al., “Platelet Derived CD154 (CD40Ligand) and Febrile Responses to Transfusion,” Lancet 357:2023-2024 (2001); Best et al., “The Interrelationship Between Thromboxane Biosynthesis, Aggregation and 5-hydroxytyptamine Secretion in Human Platelets in vitro,” Thrombosis & Haemostasis 43:3840 (1980), each of which is hereby incorporated by reference in its entirety). As shown in FIGS. 6A-B, respectively, the release of CD40L and TXB₂ was largely prevented in platelets exposed to a naturally occurring PPARγ agonist, 15d-PGJ₂, as well as to rosiglitazone, a synthetic PPARγ agonist. As shown in FIG. 11, the thrombin-induced release of PGE₂ was also substantially prevented in platelets exposed to 15d-PGJ₂ or rosiglitazone. The thrombin induced increase in platelet surface CD40L was also prevented by the PPARγ agonists as measured by flow cytometry (FIG. 7).

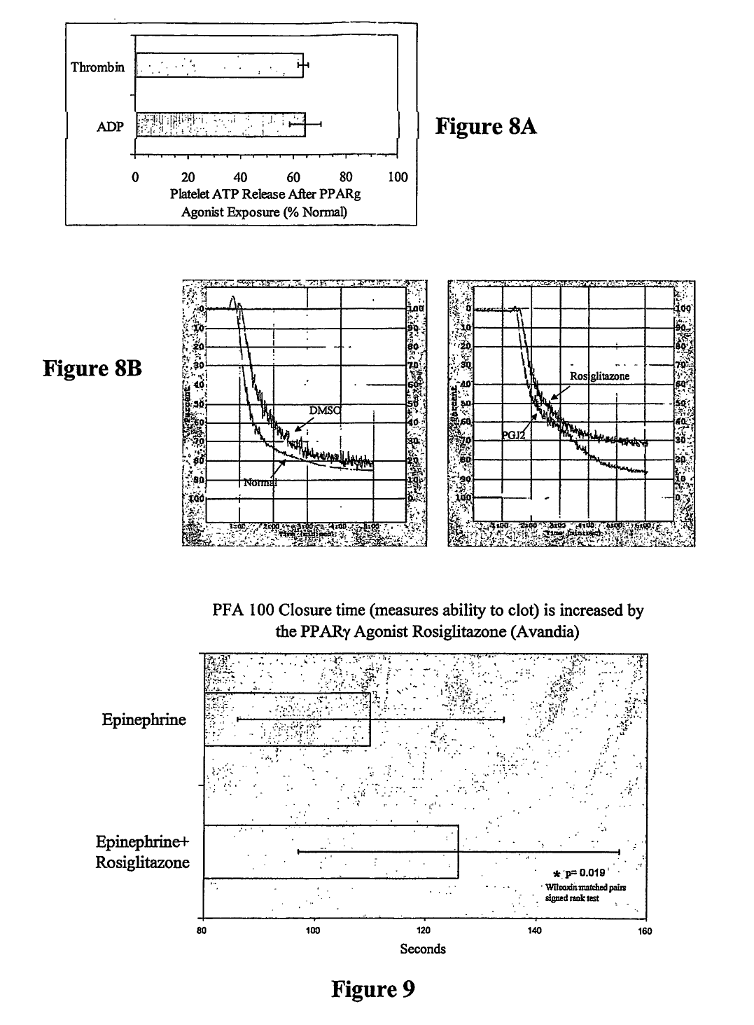

To determine if a PPARγ agonist would inhibit platelet aggregation, the natural PPARγ agonist 15d-PGJ₂ was added to PRP and aggregation and ATP release stimulated with ADP. As shown in FIG. 8B, there was a concentration dependent inhibition of platelet aggregation as shown by the results of a representative experiment. The initial slope of platelet aggregation, measured within the first 16 seconds after ADP addition, and the amplitude were significantly inhibited with 20 μM 15d-PGJ₂. The slope was 83±5% (mean±SEM) of the normal (untreated) and the amplitude of aggregation was 64±11% of normal platelets (n=7, p=0.02 for both). ATP release was also significantly inhibited by 20 μM 15d-PGJ₂ with a slope of 15±5% of normal and an amplitude of 22±10% of normal platelets (n=7, p<0.0008 for both). These findings support a role for PPARγ in down-modulating platelet activation.

### Example 7

### Platelets Express RXR Protein and Their PPARγ Has DNA Binding Ability

For PPARγ to bind DNA, current models indicate it heterodimerizes with the retinoic receptor (RXR) (Willson et al., “The PPARs: From Orphan Receptors to Drug Discovery,” J. Med. Chem. 43(4):527-550 (2000); Michalik et al., “Peroxisome Proliferation-Activated Receptors and Cancers: Complex Stories,” Nature Reviews Cancer 4:61-70 (2004), each of which is hereby incorporated by reference in is entirety). RXR was investigated in rigorously purified platelets using western blotting and an antibody (Santa Cruz SC774) that recognizes all three isoforms of the human RXR. All three platelet preparations contained RXR (see FIG. 5) which co-migrated at ˜50-55 kDa with the adipose tissue RXR (positive control). These results further reinforce that platelets do contain transcription factors and appear to possess all of the machinery to form an active PPARγ -RXR DNA binding complex.

### Example 8

### Effect of PPARγ Agonists on Platelet Activation and Clotting Time

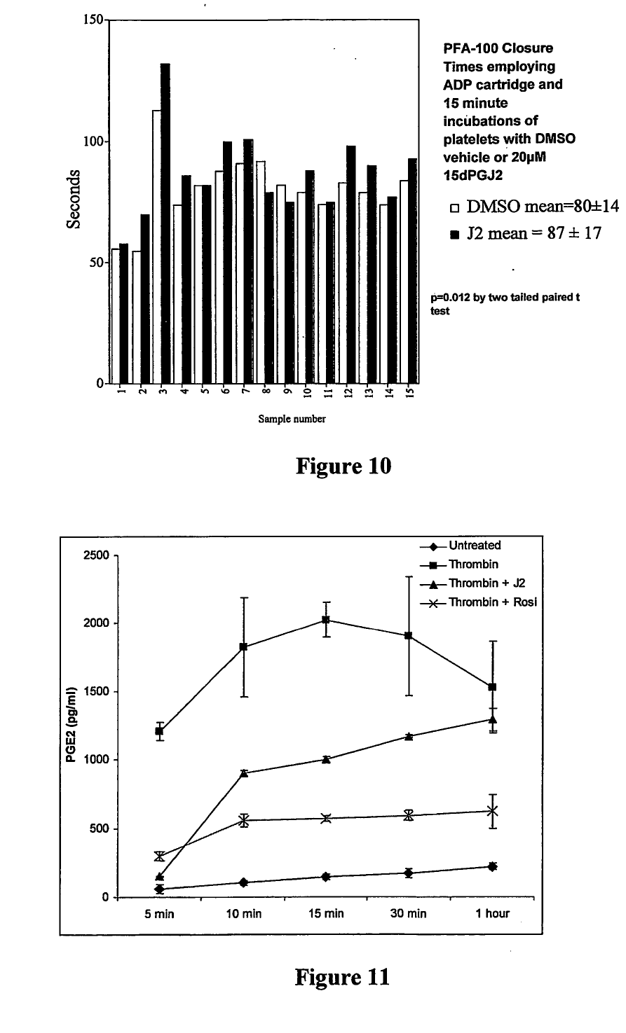

Platelet activation was induced using epinephrine or ADP, and clotting time was assessed using a PFA-100. The PFA-100 allows for an approximation of bleeding times in patients. Longer closure times equal a lower ability to form a clot. As shown in FIG. 9, the PPARγ agonist rosiglitazone (Avandia) attenuates the ability of the platelet activator epinephrine to induce a clot. Human blood was exposed to rosiglitazone or vehicle alone followed by testing in the PFA-100 activated with epinephrine. Blood exposed to rosiglitazone takes longer to form a closure (p=0.019). As shown in FIG. 10, the PPARγ agonist 15d-PGJ2 attenuates the ability of the platelet activator ADP to induce a clot. Human blood was exposed to 15d-PGJ2 or vehicle only followed by testing in the PFA-100 activated with ADP. Blood exposed to 15d-PGJ2 takes longer to form a closure (p=0.012).

### Discussion of Examples 1-8

PPARγ is believed to be expressed only by nucleated cells since it is known as a transcription factor mainly located in the nucleus (Murphy and Holder, “PPARγ Agonists: Therapeutic Role in Diabetes, Inflammation and Cancer,” Trends Pharmacol Sci. 21:469-474 (2000), each of which is hereby incorporated by reference in its entirety). However, recent studies have showed that PPARγ is not restricted to the nucleus, but is also expressed in the cytoplasm (Padilla et al., “Human B Lymphocytes and B Lymphomas Express PPAR-γ and Are Killed by PPAR-γ Agonists,” Clinical Immunology 103:22-33 (2002); Kelly et al., “Commensal Anaerobic Gut Bacteria Attenuate Inflammation by Regulating Nuclear-Cytoplasmic Shuttling of PPAR-γ and Rel A,” Nat. Immunol. 5:104-112 (2004), each of which is hereby incorporated by reference in its entirety).

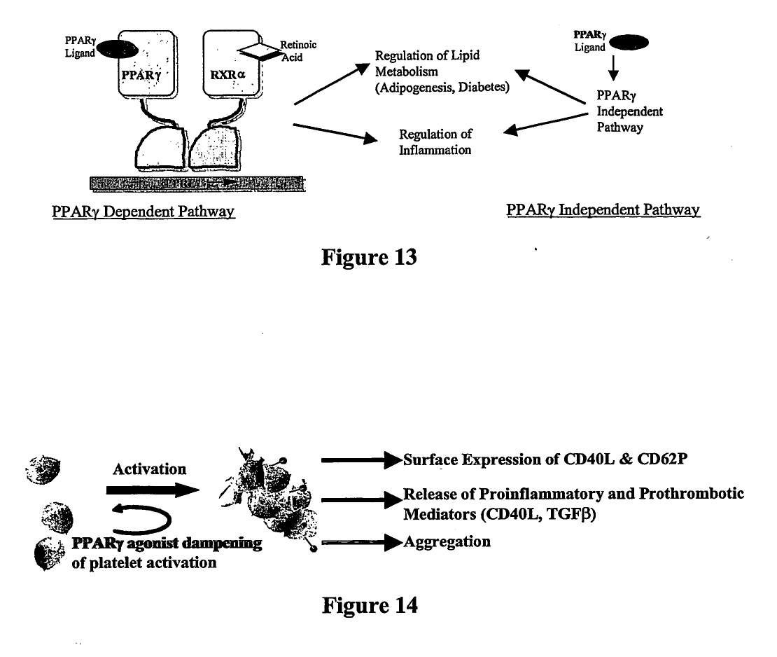

The role of PPARγ as a transcription factor, previously the only known role of PPARγ, is illustrated in FIG. 13, which shows the basic mechanism whereby PPARγ is believed to be activated by ligand binding to form an active transcriptional complex. The current models indicate that optimal DNA binding to a PPAR DNA response element occurs after ligand binding and after heterodimerization with the retinoic receptor (RXR). Note that many studies also show that some DNA binding can occur in the absence of deliberate addition of PPARγ agonist. This is likely due to the presence of low levels of endogenous PPARγ and RXR ligand in cells.

The above results provide the first evidence that the Meg-01 cell line, human bone marrow megakaryocytes and human platelets express PPARγ. The presence of PPARγ protein was demonstrated by western blotting with several different anti-PPARγ antibodies, immunocytochemistry, flow cytometry, and by peptide mapping analysis. As shown by EMSA and gel shift assay, the Meg-01 cell line and human platelets have active PPARγ protein with the ability to bind DNA. This was also shown by the TransAM PPARγ DNA binding assay. Megakaryocytes, the precursor cell of platelets, express a wide-range of mRNA encoding for a variety of bioactive mediators (Soslau et al., “Cytokine mRNA Expression in Human Platelets and a Megakaryocytic Cell Line and Cytokine Modulation of Platelet Function,” Cytokine 9:405-411 (1997), which is hereby incorporated by reference in its entirety). The Meg-01 cell line was used to test for the presence of PPARγ mRNA, and these cells do express PPARγ mRNA. Interestingly, the enucleate platelet does express some mRNAs (Gnatenko et al., “Transcript Profiling of Human Platelets Using Microarray and Serial Analysis of Gene Expression,” Blood 101:2285-2293 (2003), which is hereby incorporated by reference in its entirety). However, while we found β-actin mRNA in platelets, no PPARγ mRNA was detected. This finding supports the concept that platelets have pre-formed PPARγ protein.

The above findings that platelets contain the transcription factor PPARγ and that PPARγ agonists blunt platelet activation suggest a surprising, new non-transcriptional function for PPARγ. The exact location of PPARγ in the platelet is unknown, but based on immunohistochemical staining of platelets (FIG. 2B), it may be contained in granules with the bulk of the PPARγ being distributed throughout the platelet. Since there is abundant PPARγ permeating the platelet, it will likely have a pivotal role in regulating multiple platelet functions. Clearly, platelet PPARγ retains its DNA binding ability, which would appear to be unneeded in platelets, we therefore suggest that PPARγ must also possess other functions, which may include interactions with intracellular platelet proteins. There are several steps during platelet exocytosis wherein PPARγ could interfere, including calcium or protein kinase C signaling pathways, rearrangement of the cytoskeleton during platelet activation, or docking and fusion of granules with the plasma membrane. Further studies to determine the novel PPARγ targets in platelets will be necessary to thoroughly define the mechanism of platelet inhibition by PPARγ agonists.

Little is known about the in vivo ligands for PPARγ. One possibility in the bone marrow is that megakaryocytes generate 15d-PGJ₂, as they are known to produce its precursor PGD₂ (Greene et al., “PPAR Gamma: Observations in the Hematopoietic System,” Prostaglandins and Other Lipid Mediators 62:45-73 (2000), which is hereby incorporated by reference in its entirety). This could modulate PPARγ activity in the bone marrow. PPARγ may be involved in the differentiation and proliferation of bone marrow cells and may have additional immunologically relevant effects in erythroid, myeloid, monocytic, megakaryocytic, T and B lymphocytic, stromal and endothelial cell function. In the study described herein, we demonstrate that 15d-PGJ₂ and the thiazolidinedione class of anti-diabetic drugs, ciglitazone and rosiglitazone, play an important role in attenuating platelet activation. This was demonstrated by the ability of PPARγ agonists to block thrombin-induced platelet release of TXB₂ CD40L, and surface-associated CD40L. In addition, the PPARγ agonist 15d-PGJ₂ blunted ADP-induced platelet aggregation and ATP release. Platelets, the most numerous, enucleate and tiny blood cells, are not only essential for clotting, but are broadly involved in inflammation and pathogenesis. Platelets contain pro-inflammatory and bioactive mediators that include transforming growth factor-β, prostaglandins, thromboxanes and CD40L. TXA₂ potentiates platelet aggregation at concentrations produced by activated platelets and mediates fever and inflammation by induction of the cyclo-oxygenase-2 enzyme (Halushka et al., “Increased Platelet Thromboxane Synthesis in Diabetes Mellitus,” J. Lab. Clin. Med. 97:87-96 (1981); Caughey et al., “Up-regulation of Endothelial Cyclooxygenase-2 and Prostanoid Synthesis by Platelets: Role of Thromboxane A2,”J. Biol. Chem. 276:37839-37845 (2001), each of which is hereby is incorporated by reference in its entirety). Platelets have the highest expression of CD40L of any human cell. Platelet released CD40L, as well as CD40L expressed on the platelet surface, could activate nearby CD40 -expressing cells. Recent studies show that platelets contribute to mucosal inflammation and atherosclerosis process by expressing and releasing CD40L (Danese et al., “Platelets Trigger a CD40-Dependent Inflammatory Response in the Microvasculature of Inflammatory Bowel Disease Patients,” Gastroenterology 124:1249-1264 (2003); Heeschen et al., “Soluble CD40L in Acute Coronary Syndromes,” New Engl. J. Medicine 348:1104-1111 (2003), each of which is hereby incorporated by reference in its entirety). CD40L is now also considered a primary platelet agonist (Prasad et al., “Soluble CD40Ligand Induces β₃ Integrin Tyrosine Phosphorylation and Triggers Platelet Activation by Outside-in Signaling,” Proc. Natl. Acad. Sci. USA 100:12367-12371 (2003), which is hereby incorporated by reference in its entirety). Since platelets are activated by their own released CD40L through B₃ integrin binding, a decrease in CD40L by PPARγ ligands, could reduce platelet activation, including thrombosis (Prasad et al., “Soluble CD40Ligand Induces β₃ Integrin Tyrosine Phosphorylation and Triggers Platelet Activation by Outside-in Signaling,” Proc. Natl. Acad. Sci. USA 100: 12367-12371 (2003), which is hereby incorporated by reference in its entirety). Patients with unstable angina have higher blood concentrations of CD40L than healthy people, perhaps due to release from activated platelets (Aukrust et al., “Enhanced Levels of Soluble and Membrane-bound CD40Ligand in Patients with Unstable Angina: Possible Reflection of T Lymphocyte and Platelet Involvement in the Pathogenesis of Acute Coronary Syndromes,” Circulation 100:614-620 (1999), which is hereby incorporated by reference in its entirety). Platelet surface expression of CD40L and evidence for high CD40L levels in atheromatous plaques have served to focus attention on platelets in atherosclerosis. CD40-CD40L interaction promotes proinflammatory and proatherogenic effects in vitro and in vivo (Lutgens et al., “Both Early and Delayed Anti-CD40L Antibody Treatment Induces a Stable Plaque Phenotype,” Proc. Natl. Acad. Sci. USA 97:7464-7469 (2000), which is hereby incorporated by reference in its entirety). It has been shown that the binding of CD40L to its corresponding cellular receptors stimulates production of other pro-inflammatory cytokines, such as tumor necrosis factor-alpha and IL-1 by leukocytes and vascular endothelium (Phipps, “Atherosclerosis: The Emerging Role of Inflammation and the CD40-CD40Ligand System,” Proc. Natl. Acad. Sci. USA 97:6930-6932 (2000), which is hereby incorporated by reference in its entirety).

The pathogenesis of type 1 and type 2 diabetes involves inflammation with elevated blood levels of CD40L as in atherosclerosis (Varo et al., “Elevated Plasma Levels of the Atherogenic Mediator Soluble CD40Ligand in Diabetic Patients: A Novel Target of Thiazolidinediones,” Circulation 107:2664-2669 (2003), which is hereby incorporated by reference in its entirety). PPARγ-activating thiazolidinediones, novel insulin-sensitizing anti-diabetic agents, have been shown to exhibit anti-inflammatory effects (Jiang et al., “PPAR-gamma Agonists Inhibit Production of Monocyte Inflammatory Cytokines,” Nature 391:82-86 (1998); Ricote et al., “The Peroxisome Proliferator-activated Receptor-gamma is a Negative Regulator of Macrophage Activation,” Nature 391:79-82 (1998), each of which is hereby incorporated by reference in its entirety). Interestingly, it was recently shown that treatment of diabetic patients with a thiazolidinedione type drug decreased circulating CD40L blood levels (Varo et al., “Elevated Plasma Levels of the Atherogenic Mediator Soluble CD40Ligand in Diabetic Patients: A Novel Target of Thiazolidinediones,” Circulation 107:2664-2669 (2003); Marx et al., “Effect of Rosiglitazone Treatment on Soluble CD40L in Patients with Type 2 Diabetes and Coronary Artery Disease,” Circulation 107:1954-1957 (2003), each of which is hereby incorporated by reference in its entirety). The findings in the above examples, particularly that the PPARγ agonist 15d-PGJ₂ inhibited platelet aggregation and ATP release, support a therapeutic approach to inhibit platelet function in diabetics and other patients.

The above examples surprisingly demonstrate platelet PPARγ expression and its role in tempering platelet activation, and therefore reveal a novel target for PPARγ agonists. As illustrated in FIG. 14, the above results demonstrate that by regulating platelet activation, that PPARγ agonists reduce the ability of human platelets to release key mediators of inflammation, including thromboxanes and CD40 ligand. CD40 ligand is now viewed as a key link between platelets, inflammation and thrombosis. CD40 ligand levels are known to be elevated in diabetics and thus may be important in ongoing vascular injury, inflammation and the procoagulant phenotype in diabetics. Without being bound by belief, it is believed that We have PPARγ is expressed by healthy and diabetic platelets, is functionally active and that PPARγ ligands modulate the ability of platelets to become activated and produce mediators of inflammation that ultimately contribute to thrombosis and vascular injury. The overall biological significance of the above findings include the discovery of the direct effects of anti-diabetic PPARγ agonists on platelets that attenuate their activation, and thus may prove to be a new class of anti-thrombotic agents that will prove useful for diabetics and for others predisposed to cardiovascular disease.

Although the invention has been described in detail (both above and in the accompanying examples) for the purposes of illustration, it is understood that such detail is solely for that purpose, and variations can be made therein by those skilled in the art without departing from the spirit and scope of the invention which is defined by the following claims.

1-16. (canceled)

17. A method of inhibiting thrombosis, the method comprising:
contacting mammalian platelets with an effective amount of PPARγ, a PPARγ agonist, an RXR agonist, or a combination thereof, whereby said contacting inhibits formation of a thrombosis by the mammalian platelets.

18. The method according to claim 17 wherein the mammalian platelets are human platelets.

19. The method according to claim 18 wherein the PPARγ is human PPARγ.

20. The method according to claim 17 wherein both the PPARγ agonist and the RXR agonist contact the mammalian platelet.

21. The method according to claim 17 wherein the PPARγ agonist is selected from the group consisting of cyclopentenone class prostaglandins, lysophosphatidic acid or its derivatives, thiazolidinediones, glitazones, tyrosine-derived agonists, indole-derived agonists, and combinations thereof.

22. The method according to claim 17 wherein the RXR agonist is 9-cis-retinoic acid, trans-retinoic acid, synthetic RXR agonists, and combinations thereof.

23. The method according to claim 17 further comprising:
administering the PPARγ agonist, the RXR agonist, or an inducer of a PPARγ agonist to a mammal in a manner that provides for said contacting.

24. The method according to claim 23 wherein the inducer of a PPARγ agonist is decorin or fragments thereof, or an enzyme that catalyzes formation of prostaglandin D₂ precursor.

25. The method according to claim 23 wherein said administering is carried out via topical application, intranasal instillation, inhalation, intravenous injection, intra-arterial injection, intramuscular injection, application to a wound site, application to a surgical site, intracavitary injection, by suppository, subcutaneously, intradermally, transcutaneously, by nebulization, intraplurally, intraperitoneally, intraventricularly, intra-articularly, intra-aurally, intraocularly, or intraspinally.

26. The method according to claim 17 further comprising
administering a DNA molecule encoding PPARγ or an inducer of a PPARγ agonist to a mammal under conditions effective to cause transformation of one or more cells in a target tissue, thereby promoting expression of PPARγ or the inducer of a PPARγ agonist in the target tissue in a manner effective to cause said contacting.

27. A method of treating or preventing a thrombotic condition or disorder, the method comprising:
contacting mammalian platelets, in an individual exhibiting symptoms of or predisposed to a thrombotic condition or disorder, with an effective amount of PPARγ, a PPARγ agonist, an RXR agonist, or a combination thereof, whereby said administering inhibits platelet activation to treat or prevent the thrombotic condition or disorder.

28. The method according to claim 27 wherein the mammalian platelets are human platelets and the individual is a human.

29. The method according to claim 28 wherein the PPARγ is human PPARγ.

30. The method according to claim 27 wherein both the PPARγ agonist and the RXR agonist contact the mammalian platelet.

31. The method according to claim 27 wherein the PPARγ agonist is selected from the group consisting of cyclopentenone class prostaglandins, lysophosphatidic acid or its derivatives, thiazolidinediones, glitazones, tyrosine-derived agonists, indole-derived agonists, and combinations thereof.

32. The method according to claim 27 wherein the RXR agonist is 9-cis-retinoic acid, trans-retinoic acid, synthetic RXR agonists, and combinations thereof.

33. The method according to claim 27 further comprising:
administering PPARγ, the PPARγ agonist, the RXR agonist, or an inducer of a PPARγ agonist to the individual in a manner that provides for said contacting.

34. The method according to claim 33 wherein the inducer of a PPARγ agonist is decorin or fragments thereof, or an enzyme that catalyzes formation of prostaglandin D₂ precursor.

35. The method according to claim 33 wherein said administering is carried out via topical application, intranasal instillation, inhalation, intravenous injection, intra-arterial injection, intramuscular injection, application to a wound site, application to a surgical site, intracavitary injection, by suppository, subcutaneously, intradermally, transcutaneously, by nebulization, intraplurally, intraperitoneally, intraventricularly, intra-articularly, intra-aurally, intraocularly, or intraspinally.

36. The method according to claim 27 further comprising
administering a DNA molecule encoding PPARγ or an inducer of a PPARγ agonist to the individual under conditions effective to cause transformation of one or more cells in a target tissue, thereby promoting expression of PPARγ or the inducer of a PPARγ agonist in the target tissue in a manner effective to cause said contacting.

37. The method according to claim 27 wherein the thrombotic condition or disorder is selected from the group consisting of stroke, venous or arterial thrombosis, disseminated intravascular coagulation, myocardial infarction, pulmonary thrombo-embolism, and pulmonary hypertension.

38. A method of improving the quality of a blood product, the method comprising:
providing PPARγ, a PPARγ agonist, an RXR agonist, an inducer of a PPARγ agonist, or a combination thereof; and
introducing PPARγ, the PPARγ agonist, the RXR agonist, the inducer of a PPARγ agonist, or the combination thereof, to a blood product, wherein the PPARγ agonist, the RXR agonist, the inducer of a PPARγ agonist, or the combination thereof inhibits clotting or activation of platelets in the blood product and thereby improves the quality thereof.

39. The method according to claim 38 wherein the blood product is selected from the group consisting of whole blood, plasma, concentrated platelets, or a white blood cell product.

40. The method according to claim 38 wherein the blood product is a mammalian blood product.

41. The method according to claim 40 wherein the mammalian blood product is a human blood product.

42. The method according to claim 41 wherein the PPARγ is human PPARγ.

43. The method according to claim 38 wherein said introducing is carried out prior to storage of the blood product.

44. The method according to claim 38 wherein the blood product is whole blood and said introducing comprises:
collecting whole blood from a patient into a receptacle comprising the PPARγ agonist.

45. The method according to claim 38 wherein the blood product is plasma or concentrated platelets and said introducing comprises:
collecting whole blood from a patient;
separating the plasma or concentrated platelets from the whole blood; and
combining the PPARγ agonist, the RXR agonist, the inducer of a PPARγ agonist, or the combination thereof, with the plasma or concentrated platelets.

46. The method according to claim 38 wherein the blood product is plasma or concentrated platelets and said introducing comprises:
collecting whole blood from a patient;
combining the PPARγ agonist, the RXR agonist, the inducer of a PPARγ agonist, or the combination thereof, with the whole blood to form a treated mixture; and separating the plasma or concentrated platelets from the treated mixture.

47. The method according to claim 38 wherein the PPARγ agonist is selected from the group consisting of cyclopentenone class prostaglandins, thiazolidinediones, glitazones, tyrosine-derived agonists, indole-derived agonists, and combinations thereof.

48. The method according to claim 38 wherein the RXR agonist is selected from the group of 9-cis-retinoic acid, trans-retinoic acid, synthetic RXR agonists, and combinations thereof.

49. The method according to claim 38 wherein the inducer of a PPARγ agonist is decorin or fragments thereof, or an enzyme that catalyzes formation of prostaglandin D₂ precursor.

50. A stored blood product comprising:
a blood product that contains platelets and
an amount of PPARγ, a PPARγ agonist, an RXR agonist, an inducer of a PPARγ agonist, or a combination thereof that is effective to inhibit platelet activation.

51. The stored blood product according to claim 50 further comprising an anticoagulant.

52. The stored blood product according to claim 50 wherein the PPARγ agonist is selected from the group consisting of cyclopentenone class prostaglandins, thiazolidinediones, glitazones, tyrosine-derived agonists, indole-derived agonists, and combinations thereof.

53. The stored blood product according to claim 50 wherein the RXR agonist is selected from the group of 9-cis-retinoic acid, trans-retinoic acid, synthetic RXR agonists, and combinations thereof.

54. The stored blood product according to claim 50 wherein the inducer of a PPARγ agonist is decorin or fragments thereof, or an enzyme that catalyzes formation of prostaglandin D₂ precursor.

55. The stored blood product according to claim 50 wherein the blood product is whole blood, plasma, concentrated platelets, or a white blood cell product.

56. A method of inhibiting platelet aggregation comprising:
contacting mammalian platelets with an effective amount of PPARγ, a PPARγ agonist, an RXR agonist, or a combination thereof, whereby said contacting inhibits aggregation of the mammalian platelets.

57. The method according to claim 56 wherein the mammalian platelets are human platelets.

58. The method according to claim 57 wherein the PPARγ is human PPARγ.

59. The method according to claim 56 wherein both the PPARγ agonist and the RXR agonist contact the mammalian platelet.

60. The method according to claim 56 wherein the PPARγ agonist is selected from the group consisting of cyclopentenone class prostaglandins, lysophosphatidic acid or its derivatives, thiazolidinediones, glitazones, tyrosine-derived agonists, indole-derived agonists, and combinations thereof.

61. The method according to claim 56 wherein the RXR agonist is 9-cis-retinoic acid, trans-retinoic acid, synthetic RXR agonists, and combinations thereof.

62. The method according to claim 56 further comprising:
administering the PPARγ agonist, the RXR agonist, or an inducer of a PPARγ agonist to a mammal in a manner that provides for said contacting.

63. The method according to claim 62 wherein the inducer of a PPARγ agonist is decorin or fragments thereof, or an enzyme that catalyzes formation of prostaglandin D₂ precursor.

64. The method according to claim 62 wherein said administering is carried out via topical application, intranasal instillation, inhalation, intravenous injection, intra-arterial injection, intramuscular injection, application to a wound site, application to a surgical site, intracavitary injection, by suppository, subcutaneously, intradermally, transcutaneously, by nebulization, intraplurally, intraperitoneally, intraventricularly, intra-articularly, intra-aurally, intraocularly, or intraspinally.

65. The method according to claim 56 further comprising
administering a DNA molecule encoding PPARγ or an inducer of a PPARγ agonist to a mammal under conditions effective to cause transformation of one or more cells in a target tissue, thereby promoting expression of PPARγ or the inducer of a PPARγ agonist in the target tissue in a manner effective to cause said contacting.

66-105. (canceled)
